SEQUENCE LISTING (38 sequences)

SEQ ID NO: 1 - protein, 255 aa, Homo sapiens
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 2 - protein, 255 aa, Homo sapiens
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 3 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 4 - protein, 282 aa, Homo sapiens
MASLGQILFWSIISIIIILAGAIALIIGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 5 - protein, 256 aa, Mus musculus
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNS

SEQ ID NO: 6 - protein, 229 aa, Mus musculus
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNS

SEQ ID NO: 7 - protein, 249 aa, Mus musculus
MEWSWVFLFFLSVTTGVHSGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSG

SEQ ID NO: 8 - protein, 249 aa, Mus Musculus
MEWSWVFLFFLSVTTGVHSGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSG

SEQ ID NO: 9 - protein, 230 aa, Mus Musculus
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSG

SEQ ID NO: 10 - protein, 230 aa, Mus musculus
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSG

SEQ ID NO: 11 - protein, 129 aa, Mus musculus
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEIN

SEQ ID NO: 12 - protein, 129 aa, Mus musculus
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEIN

SEQ ID NO: 13 - protein, 156 aa, Mus musculus
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEIN

SEQ ID NO: 14 - protein, 241 aa, Homo sapiens
MEWSWVFLFFLSVTTGVHSGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 15 - protein, 241 aa, Homo sapiens
MEWSWVFLFFLSVTTGVHSGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 16 - protein, 222 aa, Homo sapiens
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 17 - DNA, 387 bp, Homo sapiens
ggcttcggcatcagtggacggcacagtatcacagtgaccaccgtcgcctccgctggcaatataggtgaggatggcatccagtcctgtacctttgagccggacatcaaactgtctgacatagtgatacaatggctgaaggagggggtgctcggtctggtacatgagtttaaggaagggaaggatgaactgtccgagcaggatgagatgttccgggggaggaccgctgtgttcgccgatcaggtaatcgtcggaaatgcaagtctcagattgaaaaatgtgcaactgactgatgctggcacgtataaatgctacattatcacaagtaagggcaaaggaaatgctaaccttgagtataaaacaggcgcattctcaatgcccgaggtcaat

SEQ ID NO: 18 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 699 bp, Mus musculus
gagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaa

SEQ ID NO: 20 - protein, 233 aa, Mus musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - protein, 482 aa, Mus musculus
MEWSWVFLFFLSVTTGVHSGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 463 aa, Mus musculus
GFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRSSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 23 - DNA, 747 bp, Mus musculus
atggagtggtcatgggtttttctgttctttcttagcgtgactacaggcgtccattcaggattcggcataagcggcaagcacttcatcacagttacaacgtttacaagtgcggggaacattggggaagatggaacattgtcatgtacatttgagccagatatcaaactcaatggaatagtaattcagtggcttaaggagggcatcaagggcctggtccacgaatttaaggaggggaaagacgatctgtctcagcagcacgagatgttcaggggcagaaccgccgtcttcgcagaccaggttgtggtaggcaacgccagtttgcggctgaaaaacgtgcagctgactgacgccggcacctacacatgctatatccggtcctctaagggcaaggggaacgctaatctcgagtacaaaacaggcgccttttctatgccagagatcaacgtggactataacgcaagctctgaaagtctgagatgcgaggcgccaaggtggttccctcagcccaccgtcgcgtgggcttcccaggtggatcaaggcgccaacttttctgaggtttctaacaccagcttcgaactgaacagcgaaaatgtgacaatgaaggtagtcagcgttctgtataacgtgaccatcaacaatacttactcctgtatgatagaaaatgatatagccaaggctacaggagatattaaagtgacggattcagaagtgaaaaggaggagtcaactgcaactcttgaatagcggc

SEQ ID NO: 24 - DNA, 1422 bp, Homo sapiens
atggaatggagctgggtatttctgtttttcctgtcagtaacgactggcgtccattcaggcttcggcatcagtggacggcacagtatcacagtgaccaccgtcgcctccgctggcaatataggtgaggatggcatccagtcctgtacctttgagccggacatcaaactgtctgacatagtgatacaatggctgaaggagggggtgctcggtctggtacatgagtttaaggaagggaaggatgaactgtccgagcaggatgagatgttccgggggaggaccgctgtgttcgccgatcaggtaatcgtcggaaatgcaagtctcagattgaaaaatgtgcaactgactgatgctggcacgtataaatgctacattatcacaagtaagggcaaaggaaatgctaaccttgagtataaaacaggcgcattctcaatgcccgaggtcaatgtcgactataatgccagcagtgaaacattgcgctgtgaagctccccgctggttcccccagccaaccgtggtctgggcctctcaggttgatcagggggctaacttttccgaggtgagcaacaccagcttcgaactcaactctgagaatgtgaccatgaaagttgtgtctgtcctgtataatgtaacaatcaacaacacttattcatgcatgattgaaaacgacatcgccaaggcaacaggtgatattaaggtaactgaatccgagatcaaacggcggtctgagcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaatga

SEQ ID NO: 25 - protein, 473 aa, Homo sapiens
MEWSWVFLFFLSVTTGVHSGFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 454 aa, Homo sapiens
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 454 aa, Homo sapiens
GFGISGRHSITVTTVASAGNIGEDGILSCTFEPDIKLSDIVILWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1449 bp, Mus musculus
atggagtggtcatgggtttttctgttctttcttagcgtgactacaggcgtccattcaggattcggcataagcggcaagcacttcatcacagttacaacgtttacaagtgcggggaacattggggaagatggaacattgtcatgtacatttgagccagatatcaaactcaatggaatagtaattcagtggcttaaggagggcatcaagggcctggtccacgaatttaaggaggggaaagacgatctgtctcagcagcacgagatgttcaggggcagaaccgccgtcttcgcagaccaggttgtggtaggcaacgccagtttgcggctgaaaaacgtgcagctgactgacgccggcacctacacatgctatatccggtcctctaagggcaaggggaacgctaatctcgagtacaaaacaggcgccttttctatgccagagatcaacgtggactataacgcaagctctgaaagtctgagatgcgaggcgccaaggtggttccctcagcccaccgtcgcgtgggcttcccaggtggatcaaggcgccaacttttctgaggtttctaacaccagcttcgaactgaacagcgaaaatgtgacaatgaaggtagtcagcgttctgtataacgtgaccatcaacaatacttactcctgtatgatagaaaatgatatagccaaggctacaggagatattaaagtgacggattcagaagtgaaaaggaggagtcaactgcaactcttgaatagcggcgagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaatga

SEQ ID NO: 29 - protein, 482 aa, Mus musculus; identical to SEQ ID NO: 21

SEQ ID NO: 30 - protein, 463 aa, Mus musculus; identical to SEQ ID NO: 22

SEQ ID NO: 31 - protein, 463 aa, Mus musculus
GFGISGKHFITVTTFTSAGNIGEDGTLSCTFEPDIKLNGIVILWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 32 - DNA, 25 bp, Artificial sequence
  Synthetic primer
gttagatagggtctcactgggtagc

SEQ ID NO: 33 - DNA, 25 bp, Artificial sequence
  Synthetic primer
cctacagccttcagtatgccagaga

SEQ ID NO: 34 - DNA, 25 bp, Artificial sequence
  Synthetic primer
agactagtgagacgtgctacttcca

SEQ ID NO: 35 - protein, 222 aa, Homo sapiens
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRS

SEQ ID NO: 36 - DNA, 387 bp, Homo sapiens; identical to SEQ ID NO: 17

SEQ ID NO: 37 - protein, 129 aa, Homo sapiens
GFGISGRHSITVTTVASAGNIGEDGIQSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVN

SEQ ID NO: 38 - protein, 129 aa, Homo sapiens
GFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVN

(19) United States
(12) Patent Application Publication
(10) Pub. No.: US 2009/0142342 A1
(43) Pub. Date: Jun. 4, 2009

(54) B7-H4 RECEPTOR AGONIST COMPOSITIONS AND METHODS FOR TREATING INFLAMMATION AND AUTO-IMMUNE DISEASES

(75) Inventor: Lieping Chen, Sparks Glencoe, MD (US)

Correspondence Address: Pabst Patent Group LLP, 1545 PEACHTREE STREET NE, SUITE 320, ATLANTA, GA, 30309, US

(73) Assignee: Johns Hopkins University

(21) Appl. No.: 12/198,009

(22) Filed: Aug. 25, 2008

Related U.S. Application Data
(63) Continuation-in-part of application No. 11/965,425, filed on Dec. 27, 2007.
(60) Provisional application No. 60/877,319, filed on Dec. 27, 2006.
(60) Provisional application No. 60/949,742, filed on Jul. 13, 2007.

(51) Int. Cl. A61K 39/395; A61K 38/02

(52) U.S. Cl. 424/134.1; 514/2; 424/130.1

(57) ABSTRACT

Compositions containing B7-H4 receptor agonists in an amount effective to reduce, inhibit, or mitigate an inflammatory response in an individual and methods for the treatment or prophylaxis of inflammatory disorders and autoimmune diseases or disorders have been developed. It has been discovered that B7-H4 receptor agonists, for example B7-H4 fusion proteins function as an agonist of the B7-H4 receptor on T cells to suppress both humoral and cellular autoimmunity activity. In one embodiment, B7-H4 fusion proteins compete with sH4 for a common receptor on T cells.

### CROSS-REFERENCE TO RELATED APPLICATIONS

This application is a continuation-in-part of pending U.S. patent application Ser. No. 11/965,425 which claims benefit of and priority to U.S. Ser. No. 60/877,319 filed on Dec. 27, 2006 and U.S. Ser. No. 60/949,742 filed on Jul. 13, 2007, all of which are incorporated by reference in their entirety.

### GOVERNMENT SUPPORT

This invention was made with Government support under Grant No. R01 CA98731, awarded by the National Institutes of Health. The Government has certain rights in this invention.

### TECHNICAL FIELD

In general, this invention relates to compositions and methods for modulating inflamatory responses, in particular to compositions and methods for treating or inhibiting inflammatory responses related to autoimmune disorders.

### BACKGROUND OF THE INVENTION

Modulating immune responses is important in the treatment of many diseases and disorders. For example, it would be advantageous to enhance an immune response in patients suffering from cancer or infection. Alternatively, it would be beneficial to inhibit or reduce an immune response in patients suffering from inflammatory conditions.

Chronic and persistent inflammation is a major cause for the pathogenesis and progression of systemic autoimmune diseases such as rheumatoid arthritis (RA) and systemic lupus erythematosus (SLE). RA is a highly inflammatory polyarthritis often leading to joint destruction, deformity and loss of function. Additive, symmetric swelling of peripheral joints is the hallmark of the disease. Extra-articular features and systemic symptoms can commonly occur and may antedate the onset of joint symptoms. Chronic pain, disability and excess mortality are unfortunate sequelae. During progression of RA, the synovial lining layer of the inflamed joints increases its thickness as a result of synovial hyperplasia and infiltration into synovial stroma by CD4+ T cells, B cells, CD8+ T cells, macrophages, dendritic cells and neutrophils (Feldmann, M. et al., Cell, 85:307-10 (1996); Moreland, L. W. et al., N Engl J Med, 337:141-7 (1997)). In SLE, the production of autoantibodies results in the deposition of immune complex in many tissues and organs including glomeruli, skin, lungs and synovium, thereby generating rheumatic lesions with characteristic chronic inflammation and tissue damage.

In several arthritis models, depletion of neutrophils resulted in a decrease of arthritis severity. The most common animal model for RA is collagen-induced arthritis (CIA) in which challenge with type II chicken collagen (CII) induces persistent chronic inflammation in all major joints of DBA/1j mice (Williams, R. O., et al., Proc Natl Acad Sci USA, 91:2762-6 (1994)). While CD4+ T cells have long been considered to play a central role in the pathogenesis of RA, there is renewed interest in addressing the pivotal role of neutrophils in initiation, progression and maintenance of RA. Massive infiltration of neutrophils in the lesions releases the proinflammatory cytokines including TNF-α, IL-1 and IL-6, which can affect the functions of neutrophils and other inflammatory cells.

An extensively studied murine model for SLE is the lpr strain, in which mutation of Fas apoptotic gene leads to spontaneous autoimmune disorders similar to human SLE. Studies in this strain recapitulate many aspects of human SLE symptoms. For example, lpr mice develop anti-chromatin, anti-DNA, and anti-IgG serum autoantibodies as well as a polyclonal increase of total immunoglobulin. Disease severity is highly dependent on genetic background. For example, MRL-lpr/lpr mice produce high levels of IgG autoantibodies to DNA and develop a severe glomerulonephritis due to deposition of immune complexes, while C57BL/6(B6)-lpr/lpr mice produce low level autoantibodies with much mild immunopathology.

Co-signal molecules, including those with costimulatory and coinhibitory functions, are important for the induction of effective immune response and for the prevention of unwanted autoimmunity. It has been shown that signals through the B7-CD28 family are major regulators of this balance and play a pivotal role in the regulation of autoimmunity. Persistence of inflammatory responses in systemic autoimmune diseases implies either an impaired coinhibitory or enhanced costimulatory functions, leading to the loss of the balance. In this regard, it is particularly interesting that autoantibodies against B7-H1, a primary coinhibitory molecule after binding to its receptor PD-1, is found in a significant proportion of RA patients and the presence of the autoantibodies is implicated in the progression of RA symptoms.

Soluble forms of B7-CD28 family molecules are also implicated in the progression of rheumatoid diseases. A recent study shows that soluble PD-1 could be detected in RA patients and the levels of soluble PD-1 are correlated with TNF-alpha concentration in synovial fluid. B7-H4 is a more recent addition to the B7 family member. B7-H4 has potent inhibitory effects on T cells through binding to a putative receptor, Cell surface B7-H4 is normally not detectable in normal tissues, although its surface expression could be upregulated on macrophages and tumor cells by inflammatory cytokines, including IL-10 and IL-6. It has been reported that B7-H4 could suppress T cell response in the presence of antigen stimulation. Soluble B7-H4 (sH4) has also been detected in ovarian cancer patients as a potential biomarker, but the mechanism of production and the function of sH4 is unknown. B7-H4 deficient mice were found to mount slightly enhanced T helper 1 type T cell responses against Leishmania major infection. Using independently generated B7-H4 knockout mice, it was demonstrated that the lack of B7-H4 led to resistance to Listeria monocytogenes infection which occurs by direct regulation of growth of neutrophil progenitors. In summary, although B7-H4 clearly plays a role in immunity, especially autoimmunity and resistance to infection, the mechanism is not clear.

Therefore, it is object of the invention to provide compositions and methods for the treatment of autoimmune disorders.

It is another object to the invention to provide compositions and methods for the treatment of inflammatory responses.

### SUMMARY OF THE INVENTION

Compositions containing B7-H4 receptor agonists in an amount effective to reduce, inhibit, or mitigate an inflammatory response in an individual and methods for the treatment or prophylaxis of inflammatory disorders and autoimmune diseases or disorders have been developed. It has been discovered that B7-H4 receptor agonists, for example B7-H4 fusion proteins function as an agonist of the B7-H4 receptor on T cells to suppress both humoral and cellular autoimmunity activity. In one embodiment, B7-H4 fusion proteins compete with sH4 for a common receptor on T cells.

Suitable B7-H4 receptor agonists include, but are not limited to, B7-H4 receptor binding agents such as antibodies, natural ligands of the B7-H4 receptor and fragments thereof capable of binding to the B7-H4 receptor and inducing or promoting signal transduction through the B7-H4 receptor, and B7-H4 fusion proteins.

In certain embodiments, neutrophil-mediated inflammation is reduced or inhibited. Representative inflammatory diseases or disorders that can be treated with one or more of the B7-H4 receptor agonists to reduce, inhibit or mitigate one or more symptoms include, but are not limited to, autoimmune diseases or disorders including rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner car disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

### BRIEF DESCRIPTION OF THE DRAWINGS

FIG. 1 is a schematic diagram showing the disruption of the B7-H4 gene. A 4.7 kb DNA fragment containing exons encoding the IgV and IgC domains of murine B7-H4 gene is substituted by a 1.7 kb fragment encoding the neomycin resistant (Neo) gene. Closed boxes represent B7-H4 coding exons. Lines between exons represent intron sequences. Open boxes represent untranslated exons. The Neo is represented by a shaded box.

FIG. 2a is a line graph of percent survival versus days post Listeria monocytogenes (LM) infection in wildtype mice (♦) or B7-H4KO mice (□). FIG. 2b is a graph of CFU/g of spleen (×10⁸) on day 2 or day 3 for wildtype mice (∘) or B7-H4KO mice (▴) infected with LM. FIG. 2c is a line graph of percent spleen granulocytes versus days post LM infection in wildtype mice (♦) or B7-H4KO mice (□) infected with LM. FIG. 2d is a bar graph of CFU/g of liver×10⁴ in three B7-H4 KO mice or littermate control i.p. injected with 150 pg Gr-1 mAb or control Rat IgG (LPS-free) 24 hours prior to Listeria infection. Mice were then i.p. injected with 3×10⁶ CFU of Listeria. Twenty-four hours post infection, mice were terminated and Listeria in liver was counted.

FIG. 3 is a bar graph of LM CFU/granulocyte versus hours post LM infection in wildtype mice (♦) or B7-H4KO mice (□).

FIG. 4 is a line graph of percent survival versus days post LM infection in RKO mice (♦) or B7-H4KO mice (□).

FIG. 5a is a line graph of CPM versus G-CSF (ng/ml) in two×10⁶ bone marrow cells of wildtype mice (♦) or B7-H4KO mice (□) plated with the indicated concentration of recombinant G-CSF for 3 days. The cultures were pulsed with ³HTdR for 18 hrs before the end of culture, harvested and counted by a scintillation counter. FIG. 5b is a panel of histograms of the dilution of CFSE in gated Gr-1+CD11b+ granulocytes analyzed by flow cytometry. Two×10⁶ of bone marrow cell from the indicated mice were labeled with CFSE and cultured for 5 days. Cells were harvested and doubly stained with Gr-1/CD11b mAb.

FIG. 6 is a line graph of CPM versus days. Two×10⁶ of bone marrow cells from normal B6 mice were plated in the 96-well plates coated with 20 μg/ml of recombinant murine B7-H4Ig (□) or murine Ig control protein (▴) in the absence (A) or presence of 0.1 ng/ml (B) or 1 ng/ml (C) of recombinant murine G-CSF. Cells were harvested on day 2-5 days as indicated. The cultures were pulsed with ³HTdR for 18 hrs before the end of culture, harvested and counted by a scintillation counter. *P<0.05.

FIG. 7a is a graph showing sH4 in sera of healthy donors (HA) (♦), RA (▴), and SLE (□) patients. FIG. 7b is a western blot showing that sH4 is present in RA patients and not in healthy donors. FIG. 7c is a graph showing the correlation between concentration of the sH4 and the severity groups 0 (▴), 1 (X), 2 (♦), and 3 (▪) of RA.

FIG. 8a is a schematic of the B7-H4V, B7-H4VC and B7-H4Ig. IgV domain; IgV, IgC domain; IgC. TM; transmembrane domain, CY; cytoplasmic domain. FIG. 8b shows a graph of percent incidence versus days after collagen injection of mice immunized with chicken type II collagen in CFA on day 0 and day 21. Three groups of mice were hydrodynamic injection with control vector (□), B7-H4V (▴) or B7-H4VC (▪) on day −1 and day 20; means±s.e.m. (n=5). FIG. 8c shows a graph of clinical score versus days after collagen injection of mice immunized with chicken type II collagen in CFA on day 0 and day 21. Three groups of mice were hydrodynamically injected with control (□), B7-H4V (▴) or B7-H4VC (▪) vector on day −1 and day 20; means±s.e.m. (n=5). FIG. 8d is a bar graph showing serum levels of anti-CII total IgG. white; control vector, gray; B7-H4V, black; B7-H4VC; means±s.d. FIG. 8e shows a line graph of counts per minute versus CII μg/ml. Whole splenocytes from CIA mice injected with control vector (□), B7-H4V (▴) or B7-H4VC (▪) on day 30 were cultured in the presence of the indicated amounts of CII for 72 hr; means±s.d. FIG. 8f shows bar graphs of supernatants of whole splenocytes after a 72 hr culture assessed for IFN-γ and IL-17 production by ELISA; means±s.d. FIG. 5g shows a line graph of incidence versus days after collagen injection of mice immunized with chicken type II collagen in CFA on day 0 and day 21. WT mice (□), B7-H4KO mice (▪); means±s.e.m. (n=5). FIG. 8h shows a line graph of clinical score of mice immunized with chicken type II collagen in CFA on day 0 and day 21. WT mice (□), B7-H4KO mice (▪); means±s.e.m. (n=5).

FIG. 9a shows a bar graph of an air pouch assay showing sH4 activates neutrophils by its dominant-negative activity. Subcutaneous air pouches were injected with LPS (50 μg). After 5 h, Gr-1+ neutrophils were quantified by flow cytometry of cells rinsed from the pouch with sterile saline. Each bar represents the average of six to eight mice in each group; means±s.d. FIG. 9b shows a line graph of incidence versus days after collagen challenge. Six groups of mice were treated with control vector and control rat IgG (▴), control vector and anti-Gr-1 Ab (□), B7-H4V and control rat IgG () and B7-H4V and anti-Gr-1 Ab (∘), B7-H4VC and control rat IgG (▪) and B7-H4VC and anti-Gr-1 Ab (▪); means±s.e.m. (n=5) FIG. 9c shows a line graph of clinical score of CIA mice versus days after collagen challenge. Six groups of mice were treated with control vector and control rat IgG (▴), control vector and anti-Gr-1 Ab (□), B7-H4V and control rat IgG () and B7-H4V and anti-Gr-1 Ab (∘), B7-H4VC and control rat IgG (▪) and B7-H4VC and anti-Gr-1 Ab (▪); means±s.e.m. (n=5).

FIG. 10a shows a line graph of the serum levels of anti-double strand DNA autoantibody in MRL-lpr/lpr mice. Four groups of mice were treated with control vector and control rat IgG (▴), control vector and anti-Gr-1 Ab (□), B7-H4VC and control rat IgG (▪) and B7-H4VC and anti-Gr-1 Ab (□); means±s.e.m. (n=5). FIG. 10b shows a line graph of the serum levels of anti-double strand DNA autoantibody in B6-lpr/lpr mice (□) or B6-lpr/lpr×B7-H4KO mice (▪); means±s.e.m. FIG. 10c shows a panel of graphs showing weight and total cell number in the spleens and peripheral lymph nodes of 24 weeks old B6-lpr/lpr mice (□) or B6-lpr/lpr×B7-H4KO mice (□). (n=5) FIG. 10d shows a graph indicating proteinuria grade of 24 weeks old B6-lpr/lpr mice (□) or B6-lpr/lpr×B7-H4KO mice (□). (n=5).

FIG. 11a shows a line graph of incidence of mice immunized with chicken type II collagen in CFA on day 0 and day 21. Three groups of mice were hydrodynamic injection with control vector (□) or B7-H4Ig (▪) on day −1 and day 20; means±s.e.m. (n=5) FIG. 11b shows a line graph of clinical score of mice immunized with chicken type II collagen in CFA on day 0 and day 21. Three groups of mice were hydrodynamic injection with control vector (□) or B7-H4Ig (▪) on day −1 and day 20; means±s.e.m. (n=5) FIG. 11c shows a bar graph of serum levels of anti-CII total IgG. white; control vector, black; B7-H4Ig; means±s.d. FIG. 11d shows a line graph of counts per minute versus CII μg/ml of whole splenocytes from CIA mice injected with control vector (□) or B7-H4Ig (▪) on day 30 were cultured in the presence or absence of the indicated amounts of CII for 72 hr; means±s.d. FIG. 11e shows bar graphs showing supernatants of whole splenocytes after a 72 hr culture assessed for IFN-γ and IL-17 production by ELISA; means±s.d.

FIG. 12 shows bar graphs of serum levels of anti-CII IgG1, IgG2a and IgG2b in CIA mice treated with control vector, B7-H4V, B7-H4VC or B7-H4Ig were measured by ELISAs in day 30; means±s.d.

FIG. 13 shows line graphs of counts per minute verses CII μg/ml indicating proliferation of splenic CD4 T cells in CIA mice injected with control vector (□), B7-H4V (▴), B7-H4VC (▪) or 37-H4Ig () on day 30 in the presence of the indicated amounts of CII for 72 hr; means±s.d.

FIG. 14 is a line graph of percent cumulative survival versus age (weeks) in MRL-lpr/lpr mice injected with control mIgG plasmid (□) or B7-H4Ig plasmid (▪) at 6, 8, 10 and 12 weeks of age. All phenotypes were analyzed at 19 weeks of age.

FIG. 15 is a line graph of IgG autoantibody titer (A₄₅₀ₙₘ) versus age (weeks) in MRL-lpr/lpr mice injected with control mIgG plasmid (□) or B7-H4Ig plasmid (▪).

FIG. 16 is a graph of proteinuria grade in MRL-lpr/lpr mice injected with control mIgG plasmid (□) or B7-H4Ig plasmid (□).

### DETAILED DESCRIPTION OF THE INVENTION

### I. Definitions

Unless otherwise defined, all technical and scientific terms used herein have the same meaning as commonly understood by one of ordinary skill in the art to which this invention pertains. All publications, patent applications, patents, and other references mentioned herein are incorporated by reference in their entirety where permissible. In case of conflict, the present specification, including definitions, will control. In addition, the materials, methods, and examples are illustrative only and not intended to be limiting.

The term “effective amount” or “therapeutically effective amount” means a dosage sufficient to provide treatment of the inflammatory response or autoimmune disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

A “fragment” of a B7-H4 polypeptide is a fragment of the polypeptide that is shorter than the full-length polypeptide. Generally, fragments will be five or more amino acids in length. An antigenic fragment has the ability to be recognized and bound by an antibody.

The terms “individual,” “individual,” “subject,” and “patient” are used interchangeably herein, and refer to a mammal, including, but not limited to, humans, rodents, such as mice and rats, and other laboratory animals.

As used herein, “operably linked” with regard to nucleic acids means incorporated into a genetic construct so that expression control sequences effectively control expression of a coding sequence of interest.

The terms “polypeptide” and “protein” are used interchangeably and mean any peptide-linked chain of amino acids, regardless of length or post-translational modification. Embodiments include B7-H4 polypeptides with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

As used herein “soluble B7-H4” or “4sH4” refers to fragments of B7-H4 that may be shed, secreted or otherwise extracted from cells that express B7-H4. Soluble fragments of B7-H4 include some or all of the extracellular domain of the B7-H4 polypeptide, and lack some or all of the intracellular and/or transmembrane domains. In one embodiment, soluble B7-H4 receptor polypeptide fragments include the entire extracellular domain of the B7-H4 polypeptide. In other embodiments, the soluble fragments of B7-H4 polypeptides include fragments of the extracellular domain. Extracellular domains of B7-H4 polypeptides can be readily determined by those of skill in the art using standard methodologies such as hydropathy plotting. In another embodiment, B7-H4 polypeptide fragments include any portion of the extracellular domain that is necessary for binding to B7-H4 receptors.

As used herein, the term “treating” includes alleviating, preventing and/or eliminating one or more symptoms associated with inflammatory responses or an autoimmune disease.

### II. Anti-Inflammatory Compositions

Compositions for inhibiting, reducing, or blocking T cell activation or proliferation are provided. In certain embodiments, the compositions include as an active agent a B7-H4 receptor agonist in an amount effective to inhibit, reduce, or decrease an inflammatory response. An exemplary inflammatory response includes, but is not limited to, neutrophil-mediated inflammatory responses.

A. B7-H4 Receptor Agonists

B7-H4 receptor agonists include compounds that increase or promote signal transduction through the B7-H4 receptor. Exemplary B7-H4 receptor agonists include, but are not limited to B7-H4 polypeptides and fragments thereof capable of promoting or inducing signal transduction through the B7-H4 receptor. Additional B7-H4 receptor agonists include antibodies and antibody fragments specific for the B7-H4 receptor, B7-H4 variant polypeptides including peptidomimetics of B7-H4, small molecule agonists, and B7-H4 fusion proteins.

1. Anti-B7-H4 Receptor Antibodies

Antibodies or antibody fragments that specifically bind to the B7-H4 receptor can be used to agonize the B7-H4 receptor. Methods of producing antibodies are well known and within the ability of one of ordinary skill in the art.

For example, monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas; A New Dimension in Biological Analyses, Plenum Press, New York, N.Y. (1980); H. Zola et al., in Monoclonal Hybridoma Antibodies: Techniques and Applications, CRC Press, 1982)).

Anti-idiotypic antibodies are described, for example, in Idiotypy in Biology and Medicine, Academic Press, New York, 1984; Immunological Reviews Volume 79, 1984; Immunological Reviews Volume 90, 1986; Curr. Top. Microbiol., Immunol. Volume 119, 1985; Bona, C. et al., CRC Crit. Rev. Immunol., pp. 33-81 (1981); Jerme, N K, Ann. Immunol. 125C:373-389 (1974); Jerne, N K, In: Idiotypes—Antigens on the Inside, Westen-Schnurr, I., ed., Editiones Roche, Basel, 1982, Urbain, J. et al., Ann. Immunol. 133D, 179-(1982); Rajewsky, K. et al., Ann. Rev. Immunol. 1:569-607 (1983).

Certain embodiments provide antibodies, both polyclonal and monoclonal, reactive with novel epitopes of the B7-H4 receptor. The antibodies may be xenogeneic, allogeneic, syngeneic, or modified forms thereof, such as humanized, single chain or chimeric antibodies. Antibodies may also be antiidiotypic antibodies specific for the idiotype of an anti-B7-H4 receptor antibody. The term “antibody” is also meant to include both intact molecules as well as fragments thereof that include the antigen-binding site and are capable of binding to a B7-H4 receptor epitope. These include Fab and F(ab′)₂ fragments which lack the Fc fragment of an intact antibody, and therefore clear more rapidly from the circulation, and may have less non-specific tissue binding than an intact antibody (Wahl et al., J. Nuc. Med. 24:316-325 (1983)). Also included are Fv fragments (Hochman, J. et al., Biochemistry, 12:1130-1135 (1973); Sharon, J. et al., Biochemistry, 15:1591-1594 (1976)). These various fragments can be produced using conventional techniques such as protease cleavage or chemical cleavage (see, e.g., Rousseaux et al., Meth. Enzymol., 121:663-69 (1986)).

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography.

The immunogen may be any immunogenic portion of the B7-H4 receptor. Preferred immunogens include all or a part of the extracellular domain of human B7-H4 receptor, where these residues contain the post-translation modifications, such as glycosylation, found on the native B7-H4. Immunogens including the extracellular domain are produced in a variety of ways known in the art, e.g., expression of cloned genes using conventional recombinant methods, isolation from cells of origin, cell populations expressing high levels of B7-H4 receptor.

The mAbs may be produced using conventional hybridoma technology, such as the procedures introduced by Kohler and Milstein, Nature, 256:495-97 (1975), and modifications thereof (see above references). An animal, preferably a mouse is primed by immunization with an immunogen as above to elicit the desired antibody response in the primed animal.

B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using the B7-H4-Ig fusion protein. Positive clones are subcloned, e.g., by limiting dilution, and the mAbs are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single mAb can be harvested by decantation, filtration, or centrifugation.

The antibody may be produced as a single chain antibody or scFv instead of the normal multimeric structure, Single chain antibodies include the hypervariable regions from an Ig of interest and recreate the antigen binding site of the native Ig while being a fraction of the size of the intact Ig (Skerra, A. et al., Science, 240: 1038-1041 (1988); Pluckthun, A. et al., Methods Enzymol., 178: 497-515 (1989); Winter, G. et al. Nature, 349; 293-299 (1991); Bird et al., Science 242:423 (1988); Huston et al. Proc. Natl. Acad. Sci. USA 85:5879 (1988); Jost C R et al., J Biol Chem. 269:26267-26273 (1994); U.S. Pat. Nos. 4,704,692, 4,853,871, 4,94,6778, 5,260,203. In a preferred embodiment, the antibody is produced using conventional molecular biology techniques.

Methods of using the antibodies to detect the presence of the epitope are described in Coligan, J. E. et al., eds., Current Protocols in Immunology, Wiley-Interscience, New York 1991 (or current edition); Butt, W. R. (ed.) Practical Immunoassay: The State of the Art, Dekker, N.Y., 1984; Bizollon, Ch. A., ed., Monoclonal Antibodies and New Trends in Immunoassays, Elsevier, N.Y., 1984; Butler, J. E., ELISA (Chapter 29), In: van Oss, C. J. et al., (eds), IMMUNOCHEMISTRY, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T. S. et al., Laboratory Techniques and Biochemistry in Molecular Biology, North Holland Publishing Company, NY, (1978) (Chapter by Chard, T., “An Introduction to Radioimmune Assay and Related Techniques”).

2. B7-H4 Fusion Proteins

Soluble fusion proteins of B7-H4 that form dimers or multimers and have the ability to crosslink B7-H4 receptor polypeptides can function as B7-H4 receptor agonists. B7-H4 fusion polypeptides disclosed herein have a first fusion partner including all or a part of a B7-H4 protein fused (i) directly to a second polypeptide or, (ii) optionally, fused to a linker peptide sequence that is fused to the second polypeptide. Preferably, fusion polypeptide chains are tandemly linked via disulfide bonds or other interchain covalent bonds. An exemplary fusion protein is described in Sica, et al., B7-H4, a molecule of the B7 family, negatively regulates T cell immunity, Immunity 18, 849-61 (2003).

The B7-H4 fusion proteins can include full-length B7-H4 polypeptides, or can contain a fragment of a full length B7-H4 polypeptide. In one embodiment, the fusion protein contains a fragment of B7-H4. As used herein, a fragment of B7-H4 refers to any subset of the polypeptide that is a shorter polypeptide of the full length protein. Useful fragments are those that retain the ability to bind to their natural ligands. A B7-H4 polypeptide that is a fragment of full-length B7-H4 typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand(s) as compared to full-length B7-H4.

One embodiment provides a fusion protein in which the first fusion partner is the extracellular domain of a B7-H4 protein or a fragment of the B7-H4 protein that binds to the B7-H4 receptor on T cells. It will be appreciated that the extracellular domain can include 1, 2, 3, 4, or 5 amino acids from the transmembrane domain. Alternatively, the extracellular domain can have 1, 2, 3, 4, or 5 amino acids removed from the C terminus, N terminus or both. B7-H4 nucleotide and protein sequence are found in GENBANK under accession number AY280972. Additionally, B7-H4 is described in U.S. Pat. No. 6,891,030 and where permissible, is incorporated by reference in its entirety. The fusion protein can contain the entire extracellular domain of B7-H4 or a fragment thereof that retains biological activity of B7-H4.

Human B7-H4 can have at least 80%, 85%, 90%, 95%, 99%, or 100% sequence identity to

| (SEQ ID NO:1) | |
|---|---|
| GFGISGRHSI TVTTVASAGN IGEDGILSCT FEPDIKLSDI VIQWLKEGVL GLVHEFKEGK | 60 |
| DELSEQDEMF RGRTAVFADQ VIVGNASLRL KNVQLTDAGT YKCYIITSKG KGNANLEYKT | 120 |
| GAFSMPEVNV DYNASSETLR CEAPRWFPQP TVVWASQVDQ GANFSEVSNT SFELNSENVT | 180 |
| MKVVSVLYNV TINNTYSCMI ENDIAKATGD IKVTESEIKR RSHLQLLNSK ASLCVSSFFA | 240 |
| LSWALLPLSP YLMLK | 255 |
| or | |
| (SEQ ID NO:2) | |
| GFGTSGRHSI TVTTVASAGN IGEDGIOSOT FEPDIKLSDI VIQWLKEGVL GLVHEFKEGK | 60 |
| DELSEQDEMF RGRTAVFADQ VIVGNASLRL INVQLTDAGT YECYIITSKG IGNANLEYIT | 120 |
| GAFSNPEVNV DYNASSETLE CEAPRWFPOP TVVWASQVDQ GANFSEVSNT SFELNSENVT | 180 |
| NKVVSVLYNV TINNTYSCMI ENDIAKATOD IKVTESEIKR RSHLQLLNSK ASLCVSSFFA | 240 |
| ISWALLPLSP YLMLK | 255 |
| or | |
| (SEQ ID NO:3) | |
| MASLGQILFW SIISIIIILA GAIALIIGFG ISGRHSITVT TVASAGNIGE DGILSCTFEP | 60 |
| DIKLSDIVIQ WLKEGVLGLV HEFKEGKDEL SEQDEMFRGR TAVFADQVIV GNASLRLKNV | 120 |
| QLTDAGTYKC YIITSKGKGN ANLEYKTGAF SMPEVNVDYN ASSETLRCEA PRWFPQPTVV | 180 |
| WASQVDQGAN FSEVSNTSFE LNSENVTMKV VSVLYNVTIN NTYSCMIEND IAKATGDIKV | 240 |
| TESEIKRRSH LQLLNSKASL CVSSFFAISW ALLPLSPYLM LK | 282 |
| or | |
| (SEQ ID NO:4) | |
| MASLGQILFW SIISIIIILA GAIALIIGFG ISGRHSITVT TVASAGNIGE DGIQSCTFEP | 60 |
| DIKLSDIVIQ WLKEGVLGLV HEFKEGKDEL SEQDEMFRGR TAVFADQVIV GNASLRLKNV | 120 |
| QLTDAGTYKC YIITSKGKGN ANLEYKTGAF SMPEVNVDYN ASSETLRCEA PRWFPQPTVV | 180 |
| WASQVDQGAN FSEVSNTSFE LNSENVTMKV VSVLYNVTIN NTYSCMIEND IAKATGDIKV | 240 |
| TESEIKRRSH LQLLNSKASL CVSSFFAISW ALLPLSPYLM LK. | 282 |

It will be appreciated that SEQ ID NOs: 3 and 4 include a signal peptide.

In a preferred embodiment, the fusion protein includes the extracellular domain of B7-H4 as shown in SEQ ID NOs:1-4 or fragment thereof fused to an Ig Fc constant region. Recombinant B7-H4Ig fusion protein can be prepared by fusing the coding region of the extracellular domain of B7-H4 to the Fc constant region of mouse IgG2a or human IgG1 as described previously (Chapoval, et al., Methods Mol. Med., 45:247-255 (2000)).

a. B7-H4 Extracellular Domain Fusion Partners

The first fusion partner of the B7-H4 fusion protein includes the extracellular domain of B7-H4, the membrane distal IgV domain and the membrane proximal IgC domain of B7-H4, or the IgV domain of B7-H4. The fusion proteins can include an endogenous signal peptide or a signal peptide from another protein or organism. It will be appreciated that the mature B7-H4 fusion protein does not include the signal peptide.

i. Murine B7-H4 Extracellular Domain Fusion Partners

In one embodiment, the first fusion partner of the fusion protein includes the membrane distal IgV domain and the membrane proximal IgC domain of murine B7-H4. The first fusion partner can have at least 80%, 85%, 90%, 95%, 99%, or 100% sequence identity to the murine amino acid sequence:

| (SEQ ID NO:5) | |
|---|---|
| MASLGQIIFW SIINIIIILA GAIALIIGFG ISGKHFITVT TFTSAGNIGE DGTLSCTFEP | 60 |
| DIKLNGIVIQ WLKEGIKGLV HEFKEGKDDL SQQHEMFRGR TAVFADQVVV GNASLRLKNV | 120 |
| QLTDAGTYTC YIRTSKGKGN ANLEYKTGAF SMPEINVDYN ASSESLRCEA PRWFFQPTVA | 180 |
| WASQVDQGAN FSEVSNTSFE LNSENVTMKV VSVLYNVTIN NTYSCMIEND IAKATGDIKV | 240 |
| TDSEVKRRSQ LQLLNS | 256 |

also referred to as B7-H4VC. It will be appreciated that the signal sequence will be removed in the mature protein. Additionally, it will be appreciated that signal peptides from other organisms can be used to enhance the secretion of the fusion protein from a host during manufacture. SEQ ID NO 6 provides the murine amino acid sequence without the signal sequence.

| (SEQ ID NO:6) | |
|---|---|
| GFGISGKHFI TVTTFTSAGN IGEDGTLSCT FEPDIKLNGI VIQWLKEGIK GLVHEFKEGK | 60 |
| DDLSQQHEMF RGRTAVFADQ VVVGNASLRL KNVQLTDAGT YTCYIRTSKG KGNANLEYKT | 120 |
| GAFSMPEINV DYNASSESLR CEAPRWFPQP TVAWASQVDQ GANFSEVSNT SFELNSENVT | 180 |
| MKVVSVLYNV TINNTYSCMI ENDIAKATGD IKVTDSEVKR RSQLQLLNS. | 229 |

In another embodiment, the first fusion partner of the fusion protein includes the membrane distal IgV domain and the membrane proximal IgC domain of murine B7-H4 having at least 80%, 85%, 90%, 95%, 99%, or 100% sequence identity to following murine sequences:

| (SEQ ID NO:7) | |
|---|---|
| MEWSWVFLFF LSVTTGVHSG ECISGKHFIT VTTFTSAGNI GEDGTLSCTF EPDIKLNGIV | 60 |
| IQWLKEGIKG LVHEFKEGKD DLSQQHEMFR GRTAVFADQV VVGNASLRLK NVQLTDAGTY | 120 |
| TCYIRSSKGK GNANLSYKTG AFSMPEINVD YNASSESLRC EAPRWFPQPT VAWASQVDQG | 180 |
| ANFSEVSNTS FELNSENVTM KVVSVLYNVT INNTYSCMIE NDIAKATGDI KVTDSEVKRR | 240 |
| SQLQLLNSG | 249 |
| or | |
| (SEQ ID NO:8) | |
| MEWSWVFLFF LSVTTGVHSG FGISGKHFIT VTTFTSAGNI GEDGTLSCTF EPDIKLNGIV | 60 |
| IQWLKEGIKG LVHEFKEGKD DLSQQHEMFR GRTAVFADQV VVGNASLRLK NVQLTDAGTY | 120 |
| TCYIRTSKGK GNANLEYKTG AFSMPEINVD YNASSESLRC EAPRWFPQPT VAWASQVDQG | 180 |
| ANFSEVSNTS FELNSENVTM KVVSVLYNVT INNTYSCMIE NDIAKATGDI KVTDSEVKRR | 240 |
| SQLGLLNSG | 249 |
| or | |
| (SEQ ID NO:9) | |
| GFGISGKHFI TVTTFTSAGN IGEDGTLSCT FEPDIKLNGI VIQWLKEGIK GLVHEFKEGK | 60 |
| DDLSQQHEMF RGRTAVFADQ VVVGNASLRL KNVQLTDAGT YTCYIRSSKG KGNANLEYKT | 120 |
| GAFSMPEINV DYNASSESLR CEAPRWFPQP TVAWASQVDQ GANFSEVSNT SFELNSENVT | 180 |
| MKVVSVLYNV TINNTYSCMI ENDIAKATGD IKVTDSEVKR RSQLQLLNSG | 230 |
| or | |
| (SEQ ID NO:10) | |
| GFGISGKHFI TVTTFTSAGN IGEDGTLSCT FEPDIKLNGI VIQWLKEGIK GLVHEFKEGK | 60 |
| DDLSQQHEMF RGRTAVFADQ VVVGNASLRL KNVQLTDAGT YTCYIRTSKG KGNANLEYKT | 120 |
| GAFSMPEINV DYNASSESLR CEAPRWFPQP TVAWASQVDQ GANFSEVSNT SFELNSENVT | 180 |
| MKVVSVLYNV TINNTYSCMI ENDIAKATGD IKVTDSEVKR RSQLQLLNSG. | 230 |

In still another embodiment, the first fusion partner of the fusion protein includes the membrane distal IgV domain of murine B7-H4 having at least 80%, 85%, 90%, 95%, 99%, 100% sequence identity to the murine amino acid sequences:

| (SEQ ID NO: 11) | |
|---|---|
| GFGISGKHFI TVTTETSAGN IGEDGTLSCT FEPDIKLNGI VIQWLKEGIK GLVHEFKEGK | 60 |
| DDLSQQHEMF RGRTAVFADQ VVVGNASLRL KNVGLTDAGT YTCYIRSSKG KGNANLEYKT | 120 |
| GAFSMPEIN | 129 |
| or | |
| (SEQ ID NO:12) | |
| GFGISGKHFI TVTTFTSAGN IGEDGTLSCT FEPDIKLNGI VIQWLKEGIK GLVHEFKEGK | 60 |
| DDLSQQHEMF RGRTAVFADQ VVVGNASLRL KNVQLTDAGT YTCYIRTSKG KGNANLEYKT | 120 |
| GAFSMPEIN. | 129 |

In another embodiment, the first fusion partner of the fusion protein includes the IgV domain of murine 37-H4. The first fusion partner can have at least 80%, 85%, 90%, 95%, 99%, or 100% sequence identity to the following murine sequences:

| (SEQ ID NO:13) | |
|---|---|
| MASLGQIIFW SIINIIIILA GAIALIIGFG ISGKHFITVT TFTSAGNIGE DGTLSCTFEP | 60 |
| DIKLNGIVIQ WLKEGIKGLV HEFKEGKDDL SQQHEMFRGR TAVFADQVVV GNASLRLKNV | 120 |
| QLTDAGTYTC YIRTSKGKGN ANLEYKTGAF SMPEIN | 156 |

also referred to as B7-H4V.

ii. Human Extracellular Domain Fusion Partners

The first fusion partner of the B7-H4 fusion protein can also be the extracellular domain of human B7-H4 or a fragment thereof. A representative ECD of human B7-H4 with the signal peptide can have at least 80%, 85%, 90%, 95%, 99%, or 100% sequence identity to the following sequences:

| Human B7-H4 ECD + Signal Peptide (amino acid) | |
|---|---|
| (SEQ ID NO:14) | |
| MEWSWVFLEF LSVTTGVHSG FGISGRHSIT VTTVASAGNI GEDGIQSCTF EPDIKLSDIV | 60 |
| IQWLKEGVLG LVHEFKEGKD ELSEQDEMFR GRTAVFADQV IVGNASLRLK NVQLTDAGTY | 120 |
| KCYIITSKGK GNANLEYKTG AFSMPEVNVD YNASSETLRC EAPRWFPQPT VVWASQVDQG | 180 |
| ANFSEVSNTS FELNSENVTM KVVSVLYNVT INNTYSCMIE NDIAKATGDI KVTESEIKRR | 240 |
| S | 241 |
| or | |
| Human B7-H4 ECD + Signal Peptide (amino acid) | |
| (SEQ ID NO:15) | |
| MEWSWVFLFF LSVTTGVHSG FGISGRHSIT VTTVASAGNI GEDGILSCTF EPDIKLSDIV | 60 |
| IQWLKEGVLG LVHEFKEGKD ELSEQDEMFR GRTAVFADQV IVGNASLRLK NVQLTDAGTY | 120 |
| KCYIITSKGK GNANLEYKTG AFSMPEVNVD YNASSETLRC EAPRWFPQPT VVWASQVDQG | 180 |
| ANFSEVSNTS FELNSENVTM KVVSVLYNVT INNTYSCMIE NDIAKATGDI KVTESEIKRR | 240 |
| S | 241 |

In another embodiment the representative ECD of human B7-H4 without the signal peptide can have at least 80%, 85%, 90%, 95%, 99%, or 100% sequence identity to the following sequence:

| Human B7-H4 ECD − Signal Peptide (amino acid) | |
|---|---|
| (SEQ ID NO:16) | |
| GFGISGRHSI TVTTVASAGN IGEDGIQSCT FEPDIKLSDI VIQWLKEGVL GLVHEFKEGK | 60 |
| DELSEQDEMF RGRTAVFADQ VIVGNASLRL KNVQLTDAGT YKCYIITSKG KGNANLEYKT | 120 |
| GAFSMPEVNV DYNASSETLR CEAPRWFPQP TVVWASQVDQ GANFSEVSNT SFELNSENVT | 180 |
| MKVVSVLYNV TINNTYSCMI ENDIAKATGD IKVTESEIKR RS | 222 |
| Human B7-H4 ECD − Signal Peptide (amino acid) | |
| (SEQ ID NO:14) | |
| GFGISGRHSI TVTTVASAGN IGEDGILSCT FEPDIKLSDI VIQWLKEGVL CLVHEFKEGK | 60 |
| DELSEQDEMF RGRTAVFADQ VIVGNASLRL KNVQLTDAGT YKCYIITSKG KGNANLEYKT | 120 |
| GAFSNPEVNV DYNASSETLR CEAPRWEPQP TVVWASQVDQ GANFSEVSNT SFELNSENVT | 180 |
| MKVVSVLYNV TINNTYSCMI ENDIAKATGD IKVTESEIKR RS | 222 |

In another embodiment, the first fusion partner of the fusion protein includes the IgV domain of human B7-H4. The first fusion partner can be encoded by a nucleotide sequence having at least 80%, 85%, 90%, 95%, 99%, or 100% sequence identity to

| Human B7-H4 IgV (nucleotide) | |
|---|---|
| (SEQ ID NO:15) | |
| ggcttcggca tcagtggacg gcacagtatc acagtgacca ccgtcgcctc cgctggcaat | 60 |
| ataggtgagg atggcatcca gtcctgtacc tttgagccgg acatcaaact gtctgacata | 120 |
| gtgatacaat ggctgaagga gggggtgctc ggtctggtac atgagtttaa ggaagggaag | 180 |
| gatgaactgt ccgagcagga tgagatgttc cgggggagga ccgctgtgtt cgccgatcag | 240 |
| gtaatcgtcg gaaatgcaag tctcagattg aaaaatgtgc aactgactga tgctggcacg | 300 |
| tataaatgct acattatcac aagtaagggc aaaggaaatg ctaaccttga gtataaaaca | 360 |
| ggcgcattct caatgcccga ggtcaat | 387 |

In another embodiment, the first fusion partner of the fusion protein includes the IgV domain of human B7-H4. The first fusion partner can have at least 80%, 85%, 90%, 95%, 99%, or 100% sequence identity to the following human sequences:

| Human B7-H4 IgV (amino acid) | |
|---|---|
| (SEQ ID NO:16) | |
| GFGISGRHSI TVTTVASAGN IGEDGIQSCT FEPDIKLSDI VIQWLKEGVL GLVHEFKEGK | 60 |
| DELSEQDEMF RGRTAVFADQ VIVGNASLRL KNVQLTDAGT YKCYIITSKG KGNANLEYKT | 120 |
| GAFSMPEVN | 129 |
| Human B7-H4 IgV (amino acid) | |
| (SEQ ID NO:16) | |
| GFGISGRHSI TVTTVASAGN IGEDGILSCT FEPDIKLSDI VIQWLKEGVL GLVHEFKEGK | 60 |
| DELSEQDEMF RGRTAVFADQ VIVGNASLRL KNVQLTDAGT YKCYIITSKG KGNANLEYKT | 120 |
| GAFSMPEVN. | 129 |

iii. B7-H4 Extracellular Domain Fragments

It will be appreciated that the B7-H4 extracellular domain can contain one or more amino acids from the signal peptide or the putative transmembrane domain of B7-H4. During secretion, the number of amino acids of the signal peptide that are cleaved can vary depending on the expression system and the host. Additionally, fragments of B7-H4 extracellular domain missing one or more amino acids from the carboxy terminus or the N terminus that retain the ability to bind to the B7-H4 receptor can be used as a fusion partner for the disclosed fusion proteins.

For example, suitable fragments of B7-H4 that can be used as a first fusion partner include, but are not limited to the following:

24-241, 24-240, 24-239, 24-238, 24-237, 24-236, 24-235

23-241, 23-240, 23-239, 23-238, 23-237, 23-236, 23-235

22-241, 22-240, 22-239, 22-238, 22-237, 22-236, 22-235

21-241, 21-240, 21-239, 21-238, 21-237, 21-236, 21-235

20-241, 20-240, 20-239, 20-238, 20-237, 20-236, 20-235,

19-241, 19-240, 19-239, 19-238, 19-237, 19-236, 19-235,

18-241, 18-240, 18-239, 18-238, 18-237, 18-236, 18-235,

17-241, 17-240, 17-239, 17-238, 17-237, 17-236, 17-235,

16-241, 16-240, 16-239, 16-238, 16-237, 16-236, 16-235, of SEQ ID NO:25. It will be appreciated that the Q at position 46 can be replaced with L.

Additional fragments include 27-249, 27-250, 27-251, 27-252, 27-253, 27-254, 27-255, 27-256, 27-257, 27-258

28-249, 28-250, 28-251, 28-252, 28-253, 28-254, 28-255, 28-256, 28-257, 28-258

29-249, 29-250, 29-251, 29-252, 29-253, 29-254, 29-255, 29-256, 29-257, 29-258

30-249, 30-250, 30-251, 30-252, 30-253, 30-254, 30-255, 30-256, 30-256, 30-257-, 30-258

of SEQ ID NOs: 3 or 4, optionally with one to five amino acids of a signal peptide attached to the N terminal end.

b. Second Fusion Partners for B7-H4 Fusion Proteins

The B7-H4 polypeptide may be fused to a second polypeptide, preferably one or more domains of an Ig heavy chain constant region, preferably having an amino acid sequence corresponding to the hinge, C_{H}2 and C_{H}3 regions of a human immunoglobulin Cγ1 chain or to the hinge, C_{H}2 and C_{H}3 regions of a murine immunoglobulin Cγ2a chain.

In one embodiment, the second polypeptide contains the hinge, C_{H}2 and C_{H}3 regions of a human immunoglobulin Cγ1 chain encoded by a nucleic acid having at least 80%, 85%, 90%, 95%, 99% or 100% sequence identity to:

| (SEQ ID NO:17) | |
|---|---|
| gagactaagt catgtgacaa gacccatacg tgcccaccct gtcccgctcc agaactgctg | 60 |
| gggggaccta gcgttttctt gttcccccca aagcccaagg acaccctcat gatctcacgg | 120 |
| actcccgaag taacatgcgt agtagtcgac gtgagccacg aggatcctga agtgaagttt | 180 |
| aattggtacg tggacggagt cgaggtgcat aatgccaaaa ctaaacctcg ggaggagcag | 240 |
| tataacagta cctaccgcgt ggtatccgtc ttgacagtgc tccaccagga ctggctgaat | 300 |
| ggtaaggagt ataaatgcaa ggtcagcaac aaagctcttc ccgccccaat tgaaaagact | 360 |
| atcagcaagg ccaagggaca accccgagag ccccaggttt acacccttcc accttcacga | 420 |
| gacgagctga ccaagaacca ggtgtctctg acttgtctgg tcaaaggttt ctatccttcc | 480 |
| gacatcgcag tggagtggga gtcaaacggg cagcctgaga ataactacaa gaccacaccc | 540 |
| ccagtgcttg atagcgatgg gagctttttc ctctacagta agctgactgt ggacaaatcc | 600 |
| cgctggcagc agggaaacgt tttctcttgt agcgtcatgc atgaggccct ccacaaccat | 660 |
| tatactcaga aaagcctgag tctgagtccc ggcaaa | 696 |

The hinge, C_{H}2 and C_{H}3 regions of a human immunoglobulin Cγ1 chain encoded by SEQ ID NO:17 has the following amino acid sequence:

| (SEQ ID NO:18) | |
|---|---|
| EPKSCDKTHT CPPCPAPELL GGPSVFLFPP KPKDTLMISR TPEVTCVVVD VSHEDFEVKF | 60 |
| NWYVDGVEVH NAKTKPREEQ YNSTYRVVSV LTVLHQDWLN GKEYKCKVSN KALPAPIEKT | 120 |
| ISKAKGQPRE PQVYTLPPSR DELTKQVSL TCLVKGFYPS DIAVEWESNG QPENNYKTTP | 180 |
| PVLDSDGSFF LYSKLTVDKS RWQQGNVFSC SVMHEALHNH YTQKSLSLSP GK | 232 |

In another embodiment, the second polypeptide contains the hinge, C_{H}2 and C_{H}3 regions of a murine immunoglobulin Cγ2a chain encoded by a nucleic acid having at least 80%, 85%, 90%, 95%, 99% or 100% sequence identity to:

| (SEQ ID NO:19) | |
|---|---|
| gagccaagag gtcctacgat caagccctgc ccgccttgta aatgcccagc tccaaatttg | 60 |
| ctgggtggac cgtcagtctt tatcttcccg ccaaagataa aggacgtctt gatgattagt | 120 |
| ctgagcccca tcgtgacatg cgttgtggtg gatgtttcag aggatgaccc cgacgtgcaa | 180 |
| atcagttggt tcgttaacaa cgtggaggtg cataccgctc aaacccagaa ccacagagag | 240 |
| gattataaca gcaccctgcg ggtagtgtcc gccctgccga tccagcatca ggattggatg | 300 |
| agcgggaaag agttcaagtg taaggtaaac aacaaagatc tgccagcgcc gattgaacga | 360 |
| accattagca agccgaaagg gagcgtgcgc gcacctcagg tttacgtcct tcctccacca | 420 |
| gaagaggaga tgacgaaaaa gcaggtgacc ctgacatgca tggtaactga ctttatgcca | 480 |
| gaagatattt acgtggaatg gactaataac ggaaagacag agctcaatta caagaacact | 540 |
| gagcctgttc tggattctga tggcagctac tttatgtact ccaaattgag ggtcgagaag | 600 |
| aagaattggg tcgagagaaa cagttatagt tgctcagtgg tgcatgaggg cctccataat | 660 |
| catcacacca caaagtcctt cagccgaacg cccgggaaa | 699 |

The hinge, C_{H}2 and C_{H}3 regions of a murine immunoglobulin Cγ2a chain encoded by SEQ ID NO:3 has the following amino acid sequence:

| (SEQ ID NO:20) | |
|---|---|
| EPRGPTIKPC PPCKCPAPNL LGGPSVFIFP PKIKDVLMIS LSPIVTCVVV DVSEDDPDVQ | 60 |
| ISWFVNNVEV HTAQTQTHRE DYNSTLRVVS ALPIQHQDWM SGKEFKCKVN NKDLPAPIER | 120 |
| TISKPKGSVR APQVYVLPPP EEEMTKKQVT LTCMVTDFMP EDIYVEWTNN GKTELNYKNT | 180 |
| EPVLDSDGSY FMYSKLRVEK KNWVERNSYS CSVVHEGLHN HHTTKSFSRT PGK | 233 |

In a preferred dimeric fusion protein, the dimer results from the covalent bonding of Cys residue in the CH regions of two of the Ig heavy chains that are the same Cys residues that are disulfide linked in dimerized normal Ig heavy chains.

C. Exemplary B7-H4 Fusion Proteins

Representative murine B7-H4Ig fusion proteins have the following amino acid sequences:

| Murine B7-H4-Ig + Signal Peptide | |
|---|---|
| (SEQ ID NO:21) | |
| MEWSWVFLFF LSVTTGVHSG FGISGKHFIT VTTFTSAGNI GEDGTLSCTF EPDIKLNGIV | 60 |
| IQWLKEGIKG LVHEFKEGKD DLSQQHEMFR GRTAVFADQV VVGNASLRLK NVQLTDAGTY | 120 |
| TCYIRSSKGK GNANLEYKTG AFSMFEINVD YNASSESLRC EAPRWFPQPT VAWASQVDQG | 180 |
| ANFSEVSNTS FELNSENVTM KVVSVLYNVT INNTYSCMIE NDIAKATGDI KVTDSEVKRR | 240 |
| SQLQLLNSGE PRGPTIKPCP PCKCPAPNLL GGPSVFIFPP KIKDVLMISL SPIVTCVVVD | 300 |
| VSEDDPDVQI SWFVNNVEVH TAQTQTHRED YNSTLRVVSA LPIQHQDWMS GKEFKCKVNN | 360 |
| KDLPAPIERT ISKPKGSVRA PQVYVLPPPE EEMTKKQVTL TCMVTDFMPE DIYVEWTNNG | 420 |
| KTELNYKNTE PVLDSDGSYF MYSKLRVEKK NWVERNSYSC SVVHEGLHNH HTTKSFSRTP | 480 |
| GK | 482 |
| Murine B7-H4-Ig - Signal Peptide | |
| (SEQ ID NO:22) | |
| GFGISGKHFI TVTTFTSAGN IGEDGTLSCT FEPDIKLNGI VIQWLKEGIK GLVHEFKEGK | 60 |
| DDLSQQHEMF RGRTAVFADQ VVVGNASLRL KNVQLTDAGT YTCYIRSSKG KGNANLEYKT | 120 |
| GAFSMPEINV DYNASSESLR CEAPRWFPQP TVAWASQVDQ GANFSEVSNT SFELNSENVT | 180 |
| MKVVSVLYNV TINNTYSCMI ENDIAKATGD IKVTDSEVKR RSQLQLLNSG EPRGPTIKPC | 240 |
| PPCKCPAPNL LGGPSVFIFP PKIKDVLMIS LSPIVTCVVV DVSEDDPDVQ ISWFVNNVEV | 300 |
| HTAQTQTHRE DYNSTLRVVS ALPIQHQDWN SGKEFKCKVN NKDLPAPIER TISKPKGSVR | 360 |
| APQVYVLPPP EEEMTKKQVT LTCMVTDFMP EDIYVEWTNN GKTELNYKNT EPVLDSDGSY | 420 |
| FMYSKLRVEK KNWVERNSYS CSVVHEGLHN HHTTKSFSRT PGK | 463 |

A representative nucleotide sequence that encodes murine B7-H4 with the signal peptide is:

| Murine B7-H4 ECD + Signal Peptide (nucleotide) | |
|---|---|
| (SEQ ID NO:23) | |
| atggagtggt catgggtttt tctgttcttt cttagcgtga ctacaggcgt ccattcagga | 60 |
| ttcggcataa gcggcaagca cttcatcaca gttacaacgt ttacaagtgc ggggaacatt | 120 |
| ggggaagatg gaacattgtc azgtacattt gagccagata tcaaactcaa tggaatagta | 180 |
| attcagtggc ttaaggaggg catcaagggc ctggtccacg aatttaagga ggggaaagac | 240 |
| gatctgtctc agcagcacga gatgttcagg ggcagaaccg ccgtcttcgc agaccaggtt | 300 |
| gtggtaggca acgccagttt gcggctgaaa aacgtgcagc tgactgacgc cggcacctac | 360 |
| acatgctata tccggtcctc taagggcaag gggaacgcta atctcgagta caaaacaggc | 420 |
| gccttttcta tgccagagat caacgtggac tataacgcaa gctctgaaag tctgagatgc | 480 |
| gaggcgccaa ggtggttccc tcagcccacc gtcgcgtggg cttcccaggt ggatcaaggc | 540 |
| gccaactttt ctgaggtttc taacaccagc ttcgaactga acagcgaaaa tgtgacaatg | 600 |
| aaggtagtca gcgttctgta taacgtgacc atcaacaata cttactcctg tatgatagaa | 660 |
| aatgatatag ccaaggctac aggagatatt aaagtgacgg attcagaagt gaaaaggagg | 720 |
| agtcaactgc aactcttgaa tagcggc | 747 |

In one embodiment the human B7-H4 fusion protein is encoded by the following nucleic acid sequence.

| (SEQ ID NO:24) | |
|---|---|
| atggaatgga gctgggtatt tctgtttttc ctgtcagtaa cgactggcgt ccattcaggc | 60 |
| ttcggcatca gtggacggca cagtatcaca gtgaccaccg tcgcctccgc tggcaatata | 120 |
| ggtgaggatg gcatccagtc ctgtaccttt gagccggaca tcaaactgtc tgacatagtg | 180 |
| atacaatggc tgaaggaggg ggtgctcggt ctggtacatg agtttaagga agggaaggat | 240 |
| gaactgtccg agcaggatga gatgttccgg gggaggaccg ctgtgttcgc cgatcaggta | 300 |
| atcgtcggaa atgcaagtct cagattgaaa aatgtgcaac tgactgatgc tggcacgtat | 360 |
| aaatgctaca tcatcacaag taagggcaaa ggaaacgcta accttgagta taaaacaggc | 420 |
| gcattctcaa tgcccgaggt caatgtcgac tataatgcca gcagtgaaac attgcgctgt | 540 |
| gctaactttt ccgaggtgag caacaccagc ttcgaactca actctgagaa tgtgaccatg | 600 |
| aaagttgtgt ctgtcctgta taatgtaaca atcaacaaca cttattcatg catgattgaa | 660 |
| aacgacatcg ccaaggcaac aggtgatatt aaggtaactg aatccgagat caaacggcgg | 720 |
| tctgagccta agtcatgtga caagacccat acgtgcccac cctgtcccgc tccagaactg | 780 |
| ctggggggac ctagcgtttt cttgttcccc ccaaagccca aggacaccct catgatctca | 840 |
| cggactcccg aagtaacatg cgtagtagtc gacgtgagcc acgaggatcc tgaagtgaag | 900 |
| tttaattggt acgtggacgg agtcgaggtg cataatgcca aaactaaacc tcgggaggag | 960 |
| cagtataaca gsacctaccg cgtggtaccc gtcttgacag tgctccacca ggactggctg | 1020 |
| aatggtaagg agtacaaatg caaggtcagc aacaaagctc ttcccgcccc aattgaaaag | 1080 |
| actatcagca aggccaaggg acaaccccgc gagccccagg tttacaccct tccaccttca | 1140 |
| cgagacgagc tgaccaagaa ccaggtgtct ctgacttgtc cggtcaaagg ttcctatcct | 1200 |
| tccgacatcg cagtggagtg ggagtcaaac gggcagcctg agaataacta caagaccaca | 1260 |
| cccccagtgc ctgatagcga tgggagcttt ttcctctaca gtaagctgac tgtggacaaa | 1320 |
| tcccgctggc agcagggaaa cgttttctct tgtagcgtca tgcatgaggc cctccacaac | 1380 |
| cattatactc agaaaagcct gagtctgagt cccggcaaat ga. | 1422 |

The human B7-H4 fusion protein encoded by SEQ ID NO:24 has the following amino acid sequence:

| (SEQ ID NO:25) | |
|---|---|
| MEWSWVFLFF LSVTTGVHSG FGISGRHSIT VTTVASAGNI GEDGIQSCTF EPDIKLSDIV | 60 |
| IQWLKEGVLG LVHEFKEGKD ELSEQDEMFR GRTAVFADQV IVGNASLRLK NVQLTDAGTY | 120 |
| KCYIITSKGK GNANLEYKTG AFSMPEVNVD YNASSETLRC EAPRWFPQPT VVWASQVDQG | 180 |
| ANFSEVSNTS FELNSENVTM KVVSVLYNVT INNTYSCMIE NDIAKATGDI KVTESEIKRR | 240 |
| SEPKSCDKTH TCPPCPAPEL LGGPSVFLFP PKPKDTLMIS RTPEVTCVVV DVSHEDPEVK | 300 |
| FNWYVDGVEV HNAKTKPREE QYNSTYRVVS VLTVLHQDWL NGKEYKCKVS NKALPAPIEK | 360 |
| TISKAKGQPR EPQVYTLPPS RDELTKNQVS LTCLVKGFYP SDIAVEWESN GQPENNYKTT | 420 |
| PPVLDSDGSF FLYSKLTVDK SRWQQGNVFS CSVMHEALHN HYTQKSLSLS PGK | 473 |

The amino acid sequence of human B7-H4 fusion protein of SEQ ID NO:25 without the signal sequence is

| (SEQ ID NO:26) | |
|---|---|
| GFGISGRHSI TVTTVASAGN IGEDGIQSCT FEPDIKLSDI VIQWLKEGVL GLVHEFKEGK | 60 |
| DELSEQDEMF RGRTAVFADQ VIVGNASLRL KNVQLTDAGT YKCYIITSKG KGNANLEYKT | 120 |
| GAFSMPEVNV DYNASSETLR CEAPRWFPQP TVVWASQVDQ GANFSEVSNT SFELNSENVT | 180 |
| MKVVSVLYNV TINNTYSCMI ENDIAKATGD IKVTESEIKR PSEPKSCDKT HTCPPCPAPE | 240 |
| LLGGPSVFLF PPKPKDTLMI SRTPEVTCVV VDVSHEDPEV KFNWYVDGVE VHNAKTKPRE | 300 |
| EQYNSTYRVV SVLTVLHQDW LNGKEYKCKV SNKALPAPIE KTISKAKGQP REPQVYTLPP | 360 |
| SRDELTKNQV SLTCLVKGFY PSDIAVEWES NGQPENNYKT TPPVLDSDGS FFLYSKLTVD | 420 |
| KSRWQQGNVF SCSVMHEALH NHYTQKSLSL SPGK. | 454 |

In another embodiment, the human B7-H4 fusion protein without the signal sequence is

| (SEQ ID NO:27) | |
|---|---|
| GFGISGRHSI TVTTVASAGN IGEDGILSCT FEPDIKLSDI VILWLKEGVL GLVHEFKEGK | 60 |
| DELSEQDEMF RGRTAVFADQ VIVGNASLRL KNVQLTDAGT YKCYIITSKG KGNANLEYKT | 120 |
| GAFSMPEVNV DYNASSETLR CEAPRWFPQP TVVWASQVDQ GANFSEVSNT SFELNSENVT | 180 |
| MKVVSVLYNV TINNTYSCMI ENDIAKATGD IKVTESEIKR RSEPKSCDKT HTCPPCFAPE | 240 |
| LLGGPSVFLF PPKPKDTLMI SRTPEVTCVV VDVSHEDPEV KFNWYVDGVE VHNAKTKPRE | 300 |
| EQYNSTYRVV SVLTVLHQDW LNGKEYKCKV SNKALPAPIE KTISKAKGQP REPQVYTLPP | 360 |
| SRDELTKNQV SLTCLVKGFY PSDIAVEWES NGQPENNYKT TPPVLDSDGS FFLYSKLTVD | 420 |
| KSRWQQGNVF SCSVMHEALH NHYTQKSLSL SPGK. | 454 |

Another embodiment provides a murine B7-H4 fusion protein encoded by the following nucleic acid sequence:

| (SEQ ID NO:28) | |
|---|---|
| atggagtggt catgggtttt tctgttcttt cttagcgtya ctacaggcgt ccattcagga | 60 |
| ttaggcataa gcggcaagca cttcatcaoa gttacaacgt ttacaagtgc ggggaacatt | 120 |
| ggggaagatg gaacattgtc atgtacattt gagccagata tcaaactcaa tggaatagta | 180 |
| attcagtggc ttaaggaggg catcaagggc ctggtccacg aatttaagga ggggaaagac | 240 |
| gatctgtctc agcagcacga gatgttcagg ggcagaaccg ccgtcttcgc agaccaggtt | 300 |
| gtggtaggca acgccagttt gcggctgaaa aacgtgcagc tgactgacgc cggcacctac | 360 |
| acatgctata tccggtcctc taagggcaag gggaacgcta atctcgagta caaaacaggc | 420 |
| gccttttcta tgccagagat caacgtggac tataacgcaa gctctgaaag tctgagatgc | 480 |
| gaggcgccaa ggtggttccc tcagcccacc gtcgcgtggg cttcccaggt ggatcaaggc | 540 |
| gccaactttt ctgaggtttc taacaccagc ttcgaactga acagcgaaaa tgtgacaatg | 600 |
| aaggtagtca gcgttctgta taacgtgacc atcaacaata cttactcctg tatgatagaa | 660 |
| aatgatatag ccaaggctac aggagatatt aaagtgacgg attcagaagt gaaaaggagg | 720 |
| agtcaactgc aactcttgaa tagcggcgag ccaagaggtc ctacgatcaa gccctgcccg | 780 |
| ccttgtaaat gcccagctcc aaatttgctg ggtggaccgt cagtctttat cttcccgcca | 840 |
| aagataaagg acgtcttgat gattagtctg agccccatcg tgacatgcgt tgtggtggat | 900 |
| gtttcagagg atgaccccga cgtgcaaatc agttggttcg ttaacaacgt ggaggtgcat | 960 |
| accgctcaaa cccagaccca cagagaggat tataacagca ccctgcgggt agtgtccgcc | 1020 |
| ctgccgatcc agcatcagga ttggatgagc gggaaagagt tcaagtgtaa ggtaaacaac | 1080 |
| aaagatctgc cagcgccgat tgaacgaacc attagcaagc cgaaagggag cgtgcgcgca | 1140 |
| cctcaggttt acgtccttcc tccaccagaa gaggagatga cgaaaaagca ggtgaccctg | 1200 |
| acatgcatgg taactgactt tatgccagaa gatatttacg tggaatggac taataacgga | 1260 |
| aagacagagc tcaattacaa gaacactgag cctgttatgg attctgatgg cagotacttt | 1320 |
| atgtaatcca aattgagggt cgagaagaag aattgggtcg agagaaacag ttatagttgc | 1380 |
| tcagtggtgc atgagggcct ccataatcat cacaccacaa agtccttcag ccgaacgccc | 1440 |
| gggaaatga | 1449 |

The amino acid sequence of murine B7-H4 fusion protein including the signal sequence encoded by SEQ ID NO:28 is

| (SEQ ID NO:29) | |
|---|---|
| MEWSWVFLFF LSVTTGVHSG FGISGKHFIT VTTFTSAGNI GEDGTLSCTF EPDIKLNGIV | 60 |
| IQWLKEGIKG LVHEFKEGKD DLSQQHEMFR GRTAVFADQV VVGNASLRLK NVQLTDAGTY | 120 |
| TCYIRSSKGK GNANLEYKTG AFSMPEINVD YNASSESLRC EAPRWFPQPT VAWASQVDQG | 180 |
| ANFSEVSNTS FELNSENVTM KVVSVLYNVT INNTYSCMIE NDIAKATGDI KVTDSEVKRR | 240 |
| SQLQLLNSGE PRGPTIKPCP PCKCPAPNLL GGPSVFIFPP KIKDVLMISL SPIVTCVVVD | 300 |
| VSEDDFDVQI SWFVNNVEVH TAQTQTHRED YNSTLRVVSA LPIQHQDWMS GKEFKCKVNN | 360 |
| KDLPAPIERT ISKPKGSVRA PQVYVLPPPE EEMTKKQVTL TCMVTDFMPE DIYVEWTNNG | 420 |
| KTELNYKNTE PVLDSDGSYF MYSKLRVEKK NWVERNSYSC SVVHEGLHNH HTTKSFSRTP | 480 |
| GK. | 482 |

The amino acid sequence of murine B7-H4 fusion protein without the signal sequence is

| (SEQ ID NO:30) | |
|---|---|
| GFGISGKHFI TVTTFTSAGN IGEDGTLSCT FEPDIKLNGI VIQWLKEGIK GLVHEFKEGK | 60 |
| DDLSQQHEMF RGRTAVFADQ VVVGNASLRL KNVQLTDAGT YTCYIRSSKG KGNANLEYKT | 120 |
| GAFSMPEINV DYNASSESLR CEAPRWFPQF TVAWASQVDQ GANFSEVSNT SFELNSENVT | 180 |
| MKVVSVLYNV TINNTYSCMI ENDIAKATGD IKVTDSEVKR RSQLQLLNSG EPRGPTIKPC | 240 |
| PPCKCPAPNL LGGPSVFIFP PKIKDVLMIS LSPIVTCVVV DVSEDDPDVQ ISWFVNNVEV | 300 |
| HTAQTQTHRE DYNSTLRVVS ALPIQHQDWN SGKEFKCKVN NKDLPAPIER TISKPKGSVR | 360 |
| APQVYVLPPP EEEMTKKQVT LTCMVTDFMP EDIYVEWTNN GKTELNYKNT EFVLDSDGSY | 420 |
| FMYSKLRVEK KNWVERNSYS CSVVHEGLHN HHTTKSFSRT PGK. | 463 |

Another embodiment provides a murine B7-H4 fusion protein without the signal sequence having the following amino acid sequence

| (SEQ ID NO:31) | |
|---|---|
| GFGISGKHFI TVTTFTSAGN IGEDGTLSCT FEPDIKLNGI VILWLKEGIK GLVHEFKEGK | 60 |
| DDLSQQHEMF RGRTAVFADQ VVVGNASLRL KNVQLTDAGT YTCYIRTSKG KGNANLEYKT | 120 |
| GAFSMPEINV DYNASSESLR CEAPRWFPQP TVAWASQVDQ GANFSEVSNT SFELNSENVT | 180 |
| MKVVSVLYNV TINNTYSCMI ENDIAKATGD IKVTDSEVKR RSQLQLLNSG EPRGPTIKPC | 240 |
| PPCKCPAPNL LGGPSVFIFP PKIKDVLMIS LSPIVTCVVV DVSEDDPDVQ ISWFVNNVEV | 300 |
| HTAQTQTHRE DYNSTLRVVS ALPIQHQDWM SGKEFKCKVN NKDLPAPIER TISKPKGSVR | 360 |
| APQVYVLPPP EEEMTKKQVT LTCMVTDFMP EDIYVEWTNN GKTELNYKNT EPVLDSDGSY | 420 |
| FMYSKLRVEK KNWVERNSYS CSVVHEGLHN HHTTKSFSRT PGK | 463 |

The disclosed fusion proteins can be isolated using standard molecular biology techniques. For example, an expression vector containing a DNA sequence encoding B7-H4Ig is transfected into 293 cells by calcium phosphate precipitation and cultured in serum-free DMEM. The supernatant is collected at 72 h and the fusion protein is purified by Protein G SEPHAROSE® columns (Pharmacia, Uppsala, Sweden).

Variants of B7-H4 can also be used to produce a fusion protein that reduces, inhibits or blocks the biological function of sH4. As used herein, a “variant” B7-H4 polypeptide contains at least one amino acid sequence alteration as compared to the amino acid sequence of the corresponding wild-type B7-H4 polypeptide (e.g., a polypeptide having the amino acid sequence set forth in Accession No. AY280972). An amino acid sequence alteration can be, for example, a substitution, a deletion, or an insertion of one or more amino acids.

Variants of B7-H4 can have the same activity, substantially the same activity, or different activity than wildtype B7-H4. Substantially the same activity means that the variant is able to suppress T cell activation.

It will be appreciated that variants of the extracellular domain of B7-H4 can have at least 80% sequence identity with the extracellular domain of wild-type B7-H4 (i.e., Accession No. AY280972), typically at least 85%, more typically, at least 90%, even more typically, at least 95% sequence identity to the extracellular domain of B7-H4. In one embodiment, the fusion protein includes the extracellular domain of B7-H4 that is identical to the extracellular domain of B7-H4 in Accession No. AY280972.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch J. Mol. Biol., 48:443-453 (1970); 2) BLOSSUM62 comparison matrix from Hentikoff and Hentikoff Proc. Natl. Acad. Sci. U.S.A., 89:10915-10919 (1992); 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions can be made using any amino acid or amino acid analog. For example, substitutions can be made with any of the naturally-occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine).

Amino acid substitutions in B7-H4 fusion proteins polypeptides may be conservative substitutions. As used herein, “conservative” amino acid substitutions are substitutions wherein the substituted amino acid has similar structural or chemical properties. “Non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The disclosed fusion proteins and variants thereof preferably compete with sH4 to inhibit the biological activity of sH4, for example by binding to a common receptor. The receptor is typically a receptor on an immune cell that binds both sH4 and B7-H4. The variants of the extracellular domain of B7-H4 include conservative variants and non-conservative variants that increase the ability to of the fusion protein to compete with sH4 and thereby reduce the biological activity of sH4.

Also provided is a dimeric or trimeric fusion protein which is a dimer or trimer of the above fusion proteins. Preferably, the chains are tandemly linked via disulfide bonds or other interchain covalent bonds.

In a preferred dimeric fusion protein, the dimer results from the covalent bonding of Cys residue in the CH regions of two of the Ig heavy chains that are the same Cys residues that are disulfide linked in dimerized normal Ig H chains.

Suitable fusion proteins may include a multimer of two or more repeats of the first fusion partner linked end to end, directly or with a linker sequence between one or more monomers.

3, Peptidomimetics

Peptidomimetics of B7-H4 polypeptides are also provided. Peptidomimetics are compounds which mimic the biological activity of peptides while offering the advantages of increased bioavailability, biostability, bioefficiency, and bioselectivity against the natural biological target of the parent peptide. Peptidomimetics have general features analogous to their parent structures, polypeptides, such as amphiphilicity. Examples of such peptidomimetic materials are described in Moore et al., Chem. Rev. 101 (12), 3893-4012 (2001). As used herein, the term “peptidomimetic” includes chemically modified peptides and peptide-like molecules that contain non-naturally occurring amino acids, peptoids, and the like. Preferred substituents in peptidomimetic B7-H4 receptor agonists include those which correspond to the backbone or side chains of naturally B7-H4 polypeptides with high affinity for the receptor. Suitable classes of eptidomimetics include, but are not limited to peptoids, retro-inverso peptides, azapeptides, urea-peptidomimetics, sulphonamide peptides/peptoids, oligoureas, oligocarbamates, N,N′-linked oligoureas, oligopyrrolinones, oxazolidin-2-ones, azatides, and hydrazino peptides.

4. Small Molecule B7-H4 Receptor Agonists

Additional B7-H4 receptor agonists include small molecule agonists. The term “small molecule” refers to compounds having a molecular weight of less than about 1,000 Daltons and are non-polypeptide or non-nucleic acid molecules. Small molecule B7-H4 receptor agonists can be obtained by screening libraries of molecules, for example combinatorial libraries of organic compounds, for binding to the B7-H4 receptor. Alternatively, small molecule B7-H4 receptor agonists can be designed based on the X-ray crystallographic structure of the B7-H4 receptor.

B. Pharmaceutical Compositions

Pharmaceutical compositions including B7-H4 receptor agonists, and vectors encoding the same are provided. Pharmaceutical compositions containing peptides or polypeptides may be administered via parenteral (intramuscular, intraperitoneal, intravenous (IV) or subcutaneous injection), transdermal (either passively or using iontophoresis or electroporation), or transmucosal (nasal, vaginal, rectal, or sublingual) routes or using bioerodible inserts and can be formulated in dosage forms appropriate for each route of administration. Compositions containing agonists of B7-H4 receptors that are not peptides or polypeptides can additionally be formulated for enteral administration.

1. Formulations for Parenteral Administration

In a preferred embodiment, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Formulations for Topical Administration

Compositions disclosed herein, including B7-H4 receptor agonist polypeptides and nucleic acids encoding them can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers.

3. Controlled Delivery Polymeric Matrices

Compositions disclosed herein, including agonists of B7-H4 receptor polypeptides may also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of agonists of B7-H4 receptor polypeptides, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer Sci., 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

4. Formulations for Enteral Administration

Agonists of B7-H4 receptor polypeptides that are not peptides or polypeptides can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect peptide in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. B7-H4 receptor agonists and antagonists can be incorporated into an inert matrix which permits release by either diffusion or leaching mechanisms, e.g., films or gums. Slowly disintigrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e, impermeable to at least pH 5.0) is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films or as capsules such as those available from Banner Pharmacaps.

### III. Methods of Manufacture

As discussed above and in the examples, polypeptide B7-H4 receptor agonists, nucleic acid constructs encoding B7-H4 receptor agonists, B7-H4 or variants thereof can be produced using standard molecular biology protocols known in the art. See for example, Molecular Cloning: A Laboratory Manual (Sambrook and Russel eds. 3^{rd} ed.) Cold Spring Harbor, N.Y. (2001). Alternatively, B7-H4, sH4, antagonists or agonists thereof, or variants there of can be isolated and purified from an individual expressing them using conventional biochemical techniques.

Nucleic acids encoding B7-H4 receptor agonist polypeptides may be optimized for expression in the expression host of choice. Codons may be substituted with alternative codons encoding the same amino acid to account for differences in codon usage between the mammal from which the B7-H4 receptor nucleic acid sequence is derived and the expression host. In this manner, the nucleic acids may be synthesized using expression host-preferred codons.

One embodiment provides nucleic acids encoding B7-H4 receptor agonists that can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A. In one embodiment, a nucleic acid molecule encoding a B7-H4 receptor agonist polypeptide is present in a vector containing nucleic acids that encode one or more domains of an Ig heavy chain constant region, preferably having an amino acid sequence corresponding to the hinge, C_{H}2 and C_{H}3 regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g. a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the disclosed B7-H4 receptor agonist polypeptides described herein.

### IV. Methods of Treating Inflammatory Responses

Chronic and persistent inflammation is a major cause of the pathogenesis and progression of systemic autoimmune diseases such as rheumatoid arthritis (RA) and systemic lupus erythematosus (SLE). sH4 acts as a decoy molecule to block endogenous B7-H4. B7-H4 inhibits cell cycle progression of T cells in the presence of antigen stimulation. B7-H4 can inhibit innate immunity by suppressing proliferation of neutrophil progenitors. It is believed that elevated levels of sH4 block the inhibitory effect of endogenous B7-H4.

Therefore, an inflammatory response can be treated by interfering with the biological activity of sH4 in vivo, for example, by administering to an individual in need thereof an effective amount of an agent that inhibits or decreases the ability of sH4 to bind to the B7-H4 receptor. Interference of sH4 biological activity can be accomplished by down regulating expression of sH4, removing sH4, conjugating sH4 with a binding agent in vivo, for example an antibody, increasing the endogenous levels of B7-H4, administering B7-H4 fusion proteins, or a combination thereof.

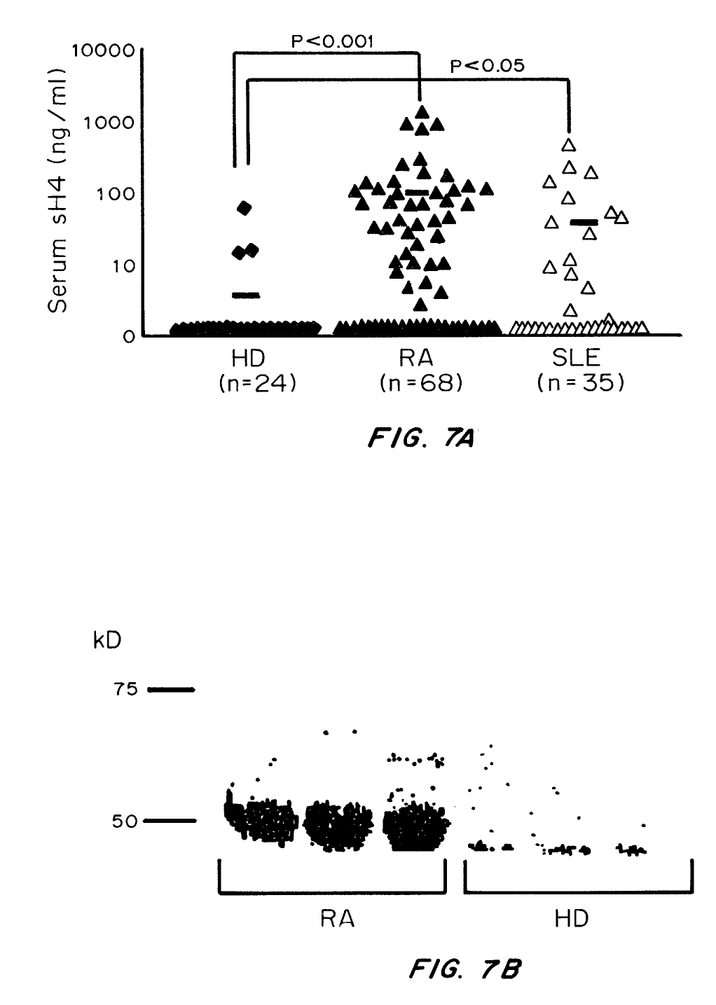

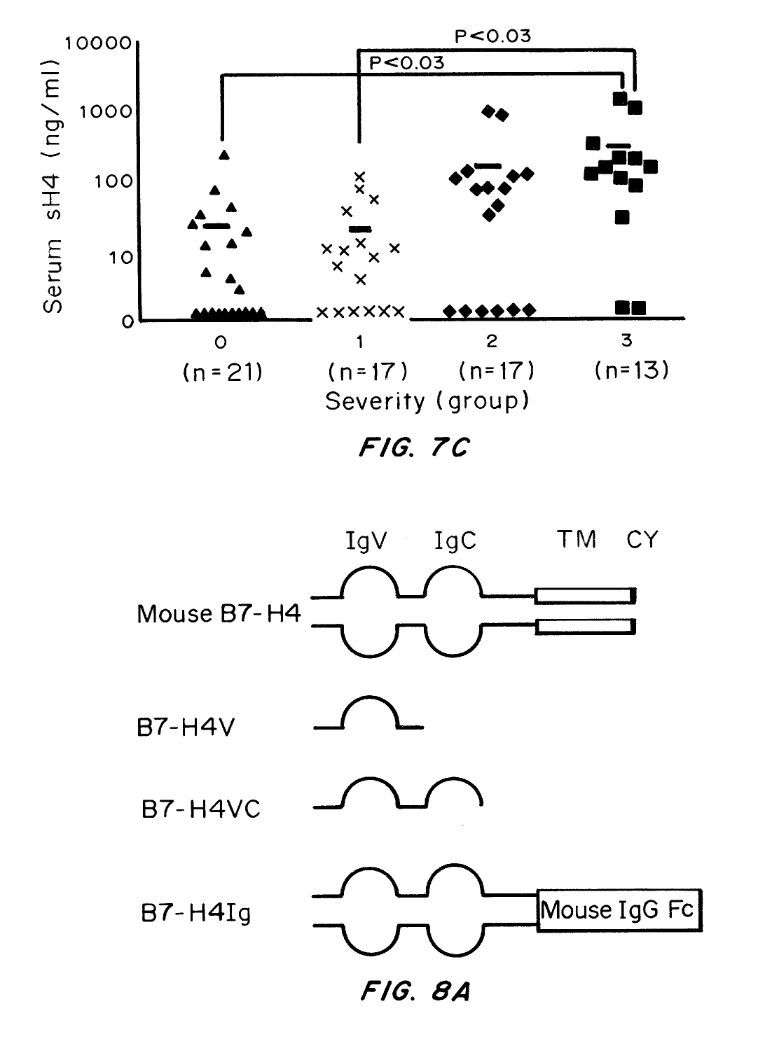

It will be appreciated that B7-H4 receptor agonists can be used alone or in combination with agents that inhibit or interfere with sH4 activity to treat inflammatory disorders in subjects. In one embodiment, B7-H4 receptor agonists are administered to a subject for the treatment of an inflammatory disease wherein the subject has little or non-detectable amounts of sH4. In another embodiment, B7-H4 receptor agonists are administered to treat one or more symptoms of an inflammatory disease in subjects having elevated levels of sH4. Elevated levels of sH4 can be determined by comparing levels of sH4 is subjects known to have an inflammatory disorder with levels of sH4 in subjects that do not have an inflammatory disorder (see FIGS. 7a and 7c).

A. Over-Expression of B7-H4

Over-expression of B7-H4 can be used to compete with endogenous sH4 and can therefore be an effective means for treating inflammatory responses and autoimmune diseases or disorders by agonizing the B7-H4 receptor. Overexpression of B7-H4 can be accomplished by stimulating endogenous B7-H4 to increase expression. Alternatively, B7-H4 can be administered as a bolus to an individual in need thereof to temporarily increase serum levels of B7-H4. B7-H4 can be administered in an amount effective to agonize the B7-H4 receptor and inhibit or reduce the activation or proliferation of T cells relative to a control.

Another method for treating an inflammatory response or autoimmune disease is by administering to an individual in need thereof a nucleic acid construct encoding B7-H4, or a functional fragment thereof. Functional fragment means a B7-H4 fragment that interferes with, inhibits or reduces sH4 biological activity.

In another embodiment, B7-H4 fusion protein can be administered to an individual in need thereof in an amount effective to reduce or inhibit inflammation or a symptom thereof. The B7-H4 fusion proteins are discussed above. Alternatively, a nucleic acid construct encoding the B7-H4 fusion can be administered to an individual in need thereof wherein the nucleic acid construct is expressed in the individual and produces B7-H4 fusion protein in amounts effective to reduce or inhibit sH4 biological function.

B. Gene Delivery

Nucleic acids encoding B7-H4 receptor agonists can be administered to an individual in need thereof in an amount effective to treat an inflammatory response or autoimmune disease. DNA delivery involves introduction of a “foreign” DNA into a cell and ultimately, into a live animal. Gene delivery can be achieved using viral vectors or non-viral vectors. Compositions and methods for delivering genes to a subject are known in the art (see Understanding Gene Therapy, Lemoine, N. R., ed., BIOS Scientific Publishers, Oxford, 2008) One approach includes nucleic acid transfer into primary cells in culture followed by autologous transplantation of the ex vivo transformed cells into the individual, either systemically or into a particular organ or tissue.

Nucleic acid therapy can be accomplished by direct transfer of a functionally active DNA into mammalian somatic tissue or organ in vivo. DNA transfer can be achieved using a number of approaches described below. These systems can be tested for successful expression in vitro by use of a selectable marker (e.g., G418 resistance) to select transfected clones expressing the DNA, followed by detection of the presence of the B7-H4 expression product (after treatment with the inducer in the case of an inducible system) using an antibody to the product in an appropriate immunoassay. Efficiency of the procedure, including DNA uptake, plasmid integration and stability of integrated plasmids, can be improved by linearizing the plasmid DNA using known methods, and co-transfection using high molecular weight mammalian DNA as a “carrier”.

Retroviral-mediated human therapy utilizes amphotropic, replication-deficient retrovirus systems (Weiss and Taylor, Cell, 82:531-533 (1995)). Such vectors have been used to introduce functional DNA into human cells or tissues, for example, the adenosine deaminase gene into lymphocytes, the NPT-II gene and the gene for tumor necrosis factor into tumor infiltrating lymphocytes.

Retrovirus-mediated gene delivery generally requires target cell proliferation for gene transfer (Bordignon et al. Science 270:470-475 (1995)). This condition is met by certain of the preferred target cells into which the present DNA molecules are to be introduced, i.e., actively growing tumor cells. Gene therapy of cystic fibrosis using transfection by plasmids using any of a number of methods and by retroviral vectors has been described by Collins et al., U.S. Pat. No. 5,240,846.

The DNA molecules encoding the B7-H4 polypeptides or fusion proteins may be packaged into retrovirus vectors using packaging cell lines that produce replication-defective retroviruses, as is well-known in the art. Additional viruses for gene delivery are described in Reynolds et al. Molecular Medicine Today, 5:25-31 (1999)).

Other virus vectors may also be used, including recombinant adenoviruses, herpes simplex virus (HSV) for neuron-specific delivery and persistence. Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms. Adeno-associated virus is also useful for human therapy.

Another vector which can express the disclosed DNA molecule and is useful in the present therapeutic setting, particularly in humans, is vaccinia virus, which can be rendered non-replicating.

In addition to naked DNA or RNA, or viral vectors, engineered bacteria may be used as vectors. A number of bacterial strains including Salmonella, BCG and Listeria monocytogenes (LM). These organisms display two promising characteristics for use as vaccine vectors: (1) enteric routes of infection, providing the possibility of oral vaccine delivery; and (2) infection of monocytes/macrophages thereby targeting antigens to professional APCs.

In addition to virus-mediated gene transfer in vivo, physical means well-known in the art can be used for direct transfer of DNA, including administration of plasmid DNA and particle-bombardment mediated gene transfer. Furthermore, electroporation, a well-known means to transfer genes into cell in vitro, can be used to transfer DNA molecules to tissues in vivo.

“Carrier mediated gene transfer” has also been described. Preferred carriers are targeted liposomes (Liu et al. Curr Med Chem, 10:1307-1315 (2003)) such as immunoliposomes, which can incorporate acylated mAbs into the lipid bilayer. Polycations such as asialoglycoprotein/polylysine may be used, where the conjugate includes a molecule which recognizes the target tissue (e.g., asialoorosomucoid for liver) and a DNA binding compound to bind to the DNA to be transfected. Polylysine is an example of a DNA binding molecule which binds DNA without damaging it. This conjugate is then complexed with plasmid DNA for transfer.

Plasmid DNA used for transfection or microinjection may be prepared using methods well-known in the art, for example using the Qiagen procedure (Qiagen), followed by DNA purification using known methods, such as the methods exemplified herein.

C. Combination Therapy

The disclosed compositions can be administered to a subject in need thereof alone or in combination with one or more additional therapeutic agents including, but not limited to immunosuppressive agents, e.g., antibodies against other lymphocyte surface markers (e.g., CD40) or against cytokines, other fusion proteins, e.g., CTLA41g, or other immunosuppressive drugs (e.g., cyclosporin A, FK506-like compounds, rapamycin compounds, or steroids), anti-proliferatives, cytotoxic agents, or other compounds that may assist in immunosuppression.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art (See, e.g. WO95122972, WO 95116691, WO 95104738, U.S. Pat. Nos. 6,015,809; 5,989,591; U.S. Pat. No. 5,567,709; 5,559,112; 5,530,006; 5,484,790; 5,385,908; 5,202,332; 5,162,333; 5,780,462; 5,120,727).

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds include, for example, those described in WO 00101385. Preferably, the language “rapamycin compound” as used herein does not include FK506-like compounds.

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

### V. Transgenic Animals

Transgenic non-human animals that do not express B7-H4 or have reduced expression are useful in screening and testing. The endogenous B7-H4 gene and alleles can be disrupted by inserting a genetic element into the gene to prevent expression. Preferably, the endogenous B7-H4 gene is deleted using homologous recombination. Representative non-human transgenic animals include mice or other rodents, sheep, goats, cows, pigs, and non-human primates.

The transgenic animals can be used to as research tools to study how B7-H4 modulates the immune system, in particular how B7-H4 suppresses immune responses. For example, the transgenic animals can be used to screen for compounds that mimic endogenous B7-H4 biological activity or for compounds that interact with soluble B7-14.

The present invention will be further understood by reference to the following non-limiting examples.

### EXAMPLES

### Example 1

### Generation of B7-H4KO Mice

Mice

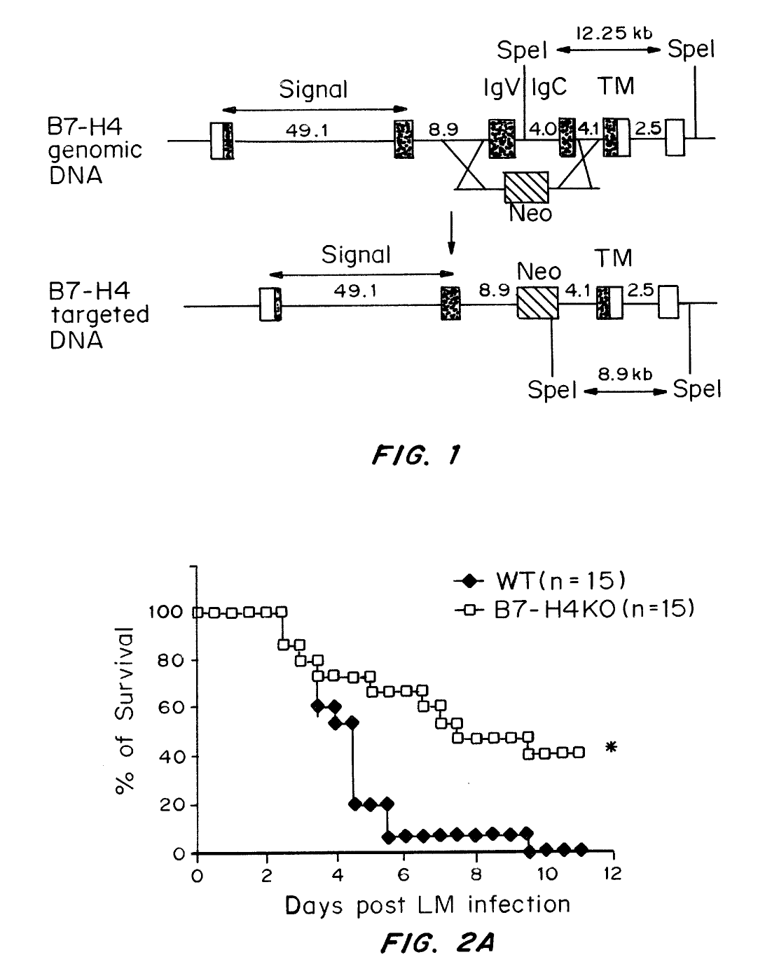

6-8-week-old C57BL16 (B6) mice were obtained from the Jackson Laboratory. RAG-1 KO mice were purchased from Taconic Farms. Both female and male mice were used for the experiments. All mice were housed under specific pathogen-free conditions in the Johns Hopkins Animal Facility with all protocols approved by the Institutional Animal Care and Use Committee. The general strategy to generate gene KO mice by homologous recombination was described by Dong, H. et al., Immunity 20:327-336 (2004); Tamada, K. et al., J Immunol., 168, 4832-4835 (2002). To generate B7-H4 KO mice, a 5.09 kb DNA fragment upstream of the IgV domain (exon 3) of the murine B7-H4 genomic DNA was PCR amplified from a 129SvJ bacterial artificial chromosome (BAC) library (Invitrogen, Carlsbad, Calif.) and was cloned into the 5′-arm position of the pKOscrambler vector NTKV-1907 (Stratagene, La Jolla, Calif.). A 5.57 kb DNA fragment downstream of the IgC domain (exon 4) of B7-H4 genomic DNA was PCR amplified from the same library and was cloned into the 3′-arm position of the same vector to generate a targeting plasmid, resulting in removing IgV and IgC domains from the B7-H4 gene (FIG. 1A). The targeting fragment containing the 5′-arm and the 3′-arm sequences of the B7-H4 gene, a positive selection marker NEO, and a negative selection marker TK was transfected into 129SvIE embryonic stem (ES) cells. ES cell transfectants underwent neomycin drug selection. The targeted clones were identified by Southern blot analysis using a 3′ external probe. Chimeric mice were produced by injection of targeted ES cells into blastocysts of B6 individuals. Heterozygous B7-H4 (+I−) mice were obtained from breeding chimeric mice with B6 mice. PCR analysis was employed to distinguish the wildtype and deficient B7-H4 allele. The sequences of the three PCR primers are: (1) 5′-GTTAGATAGGGTCTCACTGGGTAGC (SEQ ID NO:32), (2) 5′-CCTACAGCCTTCAGTATGCCAGAGA (SEQ ID NO:33), (3) 5′-AGACTAGTGAGACGTGCTACTTCCA (SEQ ID NO:34). Homozygous mice were produced by back-crossing to B6 for more than ten generations before use for further analysis. B7-H4 KO/RAG-1 KO mice were obtained by backcrossing B7-H4 KO and RAG-1 KO mice.

B7-H4KO mice were generated by homologous recombination in 129 ES cells by deleting the entire Ig V and Ig C regions of the B7-H4 gene to completely eliminate their interaction with its potential receptor. Exons encoding both the Ig V and Ig C domains of B7-H4 gene were replaced with a Neo gene cassette (FIG. 1). Targeted recombination of ES cells was confirmed by Southern blot analysis and the data from 4 independent ES clones is shown. B7-H4+ allele is predicted to have a 12.25 kb Spe1 fragment and B7-H4− allele has an 8.9 kb Spe1 fragment. The clones (2 and 3) with both fragments indicate a recombination. Chimeric male mice were derived from these ES clones by standard procedures. They were backcrossed to C57BL16 (B6) females and heterozygous mutant mice were established from two independently targeted ES clones. Heterozygous or homozygous B7-H4 mutant mice were then identified by PCR analysis of genomic DNA isolated from tail biopsies. Southern blot analysis confirmed the replacement of genomic DNA. RT-PCR analysis demonstrated B7-H4 mRNA was not expressed in livers of B7-H4-deficient mice. B7-H4KO mice develop normally and give normal litter numbers. These mice were backcrossed to the B6 background for 10 generations before they were used in studies described below.

### Example 2

### B7-H4KO Mice have Enhanced Granulocyte-Mediated Resistance to Listeria Infection

Antibodies, Recombinant Protein and Flow Cytometry Analysis

Primary and secondary antibodies against murine Gr-1 and CD11b, which are directly conjugated with FITC, PE, or APC, were purchased from BD Pharmingen (San Diego, Calif.) or eBiosciences (San Diego, Calif.). Non-conjugated primary antibodies were purified from hybridoma culture supernatant. B7-H4Ig fusion protein was prepared as described by Sica, G. L. et al., Immunity, 18:849-861 (2003). All cells were stained using standard protocols as previously described and were analyzed on a FACSCalibur flow cytometry (id). The data was analyzed with Software CellQuest (BD) or FlowJo (Tree Star, Inc., Ashland, Oreg.). For in vivo studies, mAbs were prepared and purified as previously described (id). Anti-NK1.1 hybridoma (PK136) and anti-IFN-γ hybridoma (R4-6A2) were purchased from ATCC. Anti-Gr-1 hybridoma (RB6-8C5) was a generous gift from Dr. Hans Schreiber in University of Chicago. Control mouse IgG, rat IgG, and hamster IgG were purchased from Sigma (St. Louis, Mo.) and further purified as previously described (id). Carrageenan was purchased from Sigma. All cell culture media and antibiotics were purchased from Cellgro (Herndon, Va.). Fetal bovine serum (FBS) was from Hyclone (Logan, Utah).

### Listeria Infection and Colony Counting

Listeria monocytogenes strain DP-L4056 was kindly provided by Dr. Thomas W. Dubensky Jr. from Cerus Corp. To prepare Listeria stock, Listeria cells were grown in DlFCO Listeria Enrichment Broth (Becton Dickinson Co., Sparks, Md.) to 0.8-1 at OD600 nm. Culture was harvested by centrifugation and was washed twice with PBS. Pellets were then re-suspended in stock solution (PBS with 15-20% glycerol) and aliquoted to 200 μl per microtube for storage at −80° C. The colony-forming units (CFU) of Listeria stock were determined by counting colonies of series dilutions of the aliquots growing on BBL CHROMagar Listeria plates (Becton Dickinson Co., Sparks, Md.). Prior to infection, Listeria stock was thawed and diluted in PBS to appropriate concentration of CFU/ml and applied to mice or cells as indicated. Mice 6-8 weeks old were infected by intraperitoneal (i.p.) or intravenous (i.v.) injection of indicated CFU of Listeria. At indicated time points post-infection, a piece of mouse liver or spleen was cut, weighed, and ground in PBS. The liver suspension was plated on BBL CHROMagar Listeria plates or on agar plates of Listeria Enrichment Broth. Colonies were counted 2 days post plating, and adjusted to CFUIg of liver or spleen.

Listeria Infection of Granulocytes In Vitro.

Granulocytes were isolated similar to the methods described by Chen, L. Y. et al., Hum. Mol. Genet, 12:2547-2558 (2003). Briefly, mice were injected i.p. with 3% thioglycollate broth. Four to five hours post injection, peritoneal cavities of each mouse were washed with 5 ml PBS and cells were harvested by centrifugation. By this method, more than 90% harvested cells are Gr-1⁺CD11b+ granulocyte. 1×10⁶ granulocytes were incubated with 1×10⁸ CFU of LM for 10 min at 37° C. The cultures were terminated by adding Penicillin-Streptomycin (Cellgro). Subsequently, cells were harvested by centrifugation, plated in 96-well plates. The plates were incubated at 37° C. and harvested at indicated time points. Cells were lysed immediately by resuspending in 1 ml of sterile water. Cell lysates or diluted cell lysates were plated on agar plates of Listeria Enrichment Broth for colony counting.

Respiratory burst and phagocytosis of Granulocytes.

Granulocyte phagocytic activity and oxidative burst activity were measured as described by Radsak, M. P., et al., J. Immunol., 172:4956-4963 (2004); Radsak, M. P. et al., Blood, 101:2810-281 5 (2003). Briefly, 1×10⁶ granulocytes were incubated with 5×10⁷ of red-fluorescent micro-beads (FLUORESBRITE® Polychromatic Red 1.0 Micron Microspheres, Polysciences, Inc. Warrington, Pa.) and 25 μM of DCFH-DA (2′,7′,-dihydrochlorofluoresein diacetate, Sigma-Aldrich) for 30-60 min at 37° C. Cells were washed twice with FACS buffer (1% FBS in PBS) and fixed in 1% paraformaldehyde in PBS. Analysis was performed by flow cytometry.

Pathology

The method for tissue processing and staining was described by Dong, H. et al, Nature Med 8:793-800 (2002). Briefly, spleen specimens of 6-8 week old mice were embedded in OCT compound (Sakura Finetek USA, Torrance, Calif.) and frozen at −80° C. Frozen tissues were sliced, mounted and stained with 5 μg/ml Gr-1-biotin antibody. ABC peroxidase (Vector laboratories, Inc., Burlingame, Calif.) and DAB peroxidase substrate (Sigma-Aldrich, St. Louis, Mo.) were then applied to slides according to the company protocols. Finally, hematoxylin solution was used to stain Gr-1 negative cells.

Results

B7-H4KO mice display normal numbers and ratios of T, B, NK, NKT cells, and macrophages. There are no obvious alterations in T cell responses, judged by in vitro proliferation of purified T cells by CD3 cross-linking, allogeneic antigen stimulation, or cytolytic T cell response to alloantigens. These results indicate that polyclonal T cell responses to antigens are not impaired in B7-H4KO mice. Consistent with these in vitro findings, it was also found that B7-H4KO mice have normal responses to Con-A induced hepatitis (Dong, H. et al., Immunity, 20327-336 (2004)), hapten-induced hypersensitivity (Tsushima, F. et al. Eur. J. Immunol., 33:2773-2782 (2003)), and OVA-induced airway inflammation (Kamata, T. et al., J. Clin. Invest., 111:109-119 (2003)). B7-H4-deficient mice were also found to be comparable to wild-type mice in OT-I and OT-II cell expansion to OVA proteins (Sica, G. L. et al., Immunity, 18849-861 (2003)), CD4-Vβ8.118.2 T cell expansion to superantigens (Tamada, K. et al., J Immunol., 168:4832-4835 (2002)), and CTL activities to allogeneic antigens in vivo (Tamada, K. et al, Nature Med, 6:283-289 (2000)). Normal B cell responses were also observed after immunization by TNP-KLH (Tamura, H. et al., Blood 97:1809-1816 (2001)). B7-H4KO mice do not develop spontaneous autoimmune diseases up to 1.5 years in SPF condition.

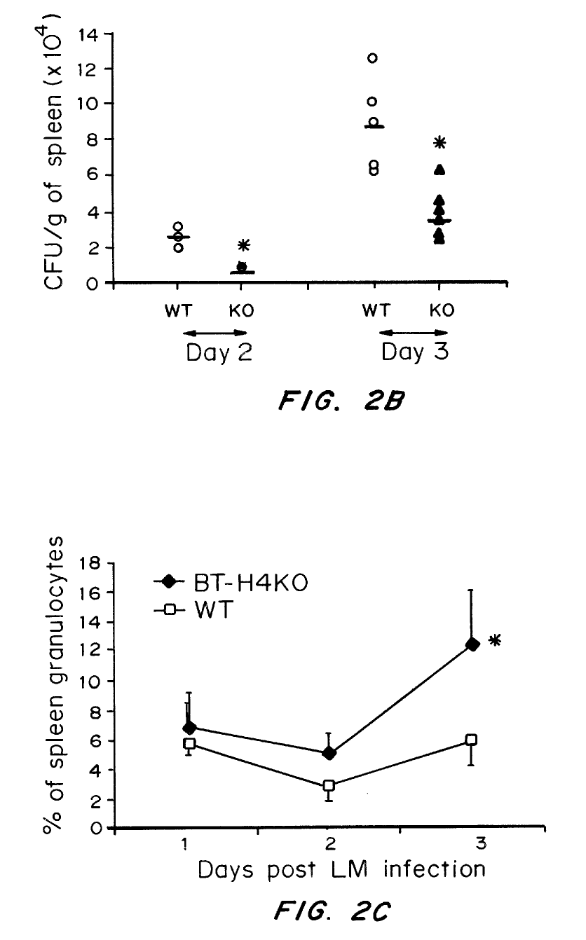

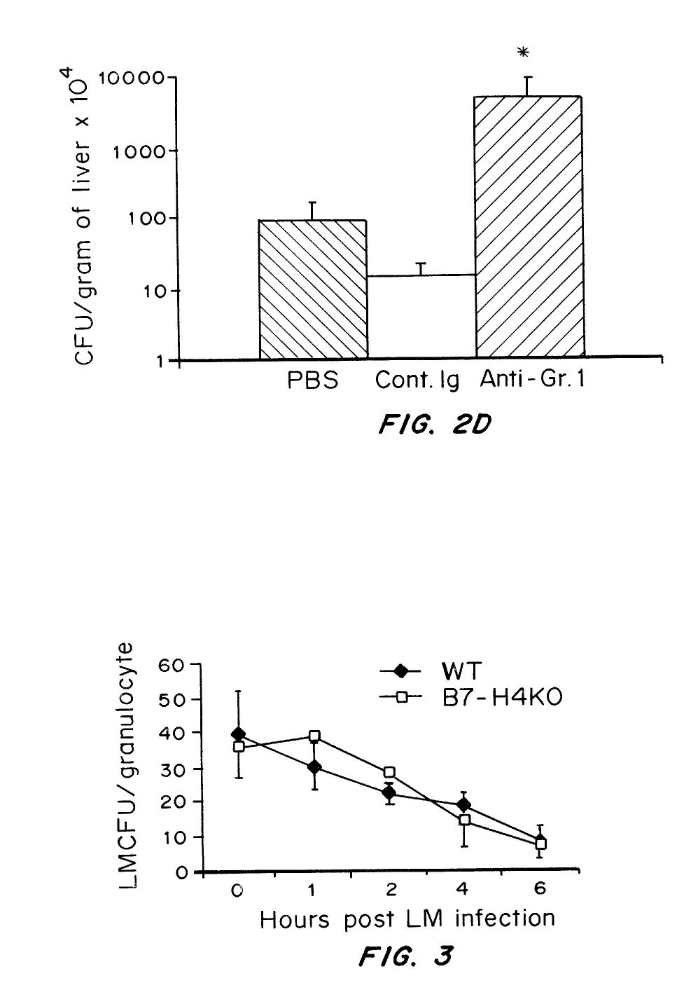

While the data indicates that B7-H4 plays a minimal role in antigen-driven T and B cell responses in assays, these responses were conducted in the absence of active infection, which usually requires a much more sophisticated coordination between innate and adaptive immunity. To test this possibility, the effect of B7-H4 ablation was evaluated in mice infected with Listeria monocytogenes (LM) to examine whether B7-H4 contributes to immunity against infection. Mice were challenged with an intra-peritoneal dose (i.p.) (2×10⁶ CFU) of LM sufficient to induce lethality. The survival of these mice was then subsequently evaluated. B7-H4KO mice were significantly more resistant to LM infection: B7-H4KO mice survived much longer than their wild-type (WT) littermates and up to 40% of mice cleared bacteria and lived indefinitely, while all littermates died around day 9 (FIG. 2a). This effect is correlated with decreased Listeria numbers in the spleens (FIG. 2b) and liver in B7-H4KO mice. Interestingly, the majority of mice were dead within 3-4 days, time points at which adaptive immunity is usually not yet developed. The results thus suggest a role of B7-H4 in altering the context of the innate immune response.

To address mechanisms of this resistance, the cell compositions of both innate and adaptive immunity were examined. The mice were infected with Listeria and T, B, NK, macrophages and granulocytes in peripheral blood and in lymphoid organs were examined by specific mAb. Although there were no significant differences in NK, macrophages, T cells, and B cells within the first 3 days after LM infection, significantly more granulocytes in spleens were found from LM-infected B7-H4KO mice than identically infected WT littermates at day 3 upon infection (FIG. 2c). Similar results were also obtained in granulocytes isolated from livers and in peripheral blood after infection. In uninfected B7-H4KO mice, however, granulocyte numbers were within normal range of WT controls. The results indicate that the role of B7-H4 is to inhibit granulocyte responses during LM infection.

To determine if granulocytes are required for the resistance of LM infection in B7-H4KO mice, granulocytes were depleted by inoculation of Gr-1 mAb. Injection of Gr-I mAb led to rapid decline of granulocytes to undetectable levels at day 2 in spleens. Depletion of Gr-I and granulocytes led to a significant increase of LM load in livers from B7-KO mice, in comparison with those treated with either PBS or isotype-matched control mAb (FIG. 2d). Depletion of NK cells by NKI.I mAb did not affect colony formation of LM in liver, while depletion of macrophages by carrageenan increased LM colonies to a moderate but less significant level as compared to Gr-I cell depletion. The results thus show that Gr-I and granulocytes play a critical role in the resistance to LM infection in the absence of B7-H4.

Whether B7-H4-deficient granulocytes have modified functionalities were determined by co-culture of purified granulocytes and LM. B7-H4-deficient granulocytes display normal uptake and growth inhibition of LM in culture system (FIG. 3). In addition, respiratory burst and phagocytosis by B7-H4KO granulocytes are also normal, indicating B7-H4KO granulocytes are functionally indifferent from WT granulocytes. Therefore, increased resistance to LM infection in B7-H4KO mice is likely caused by an increased number, not increased functional capacity of granulocytes.

### Example 3

### Granulocyte-Mediated Innate Resistance in B7-H4KO Mice is Independent of Adaptive Immunity

Activated and memory T cells are important components in the immunity against LM (Nathan, C. Nature Rev. Immunol., 6:173-182 (2006)). While the data supports that resistance of B7-H4KO mice to LM infection requires granulocytes, it is unknown whether adaptive immunity also contributes to this resistance. Because increased granulocyte numbers post-LM infection was a major phenotype found in B7-H4KO mice, the responses of B7-H4KO mice to LM infection were explored in the absence of adaptive immunity. B7-H4KO mice were backcrossed to the RAG-1 KO background to eliminate T and B cells.

Results

Unlike RAG-1 KO (RKO) mice, which possess small spleens, B7-H4/RAG-1 double KO (DKO) mice display enlarged spleens. The spleen sizes of DKO mice are similar to those of WT and B7-H4KO mice in B6 background. Further analysis of cell components in spleen, peripheral blood, liver, and bone marrow revealed that Gr1+ CD11b+ granulocytes increased dramatically.

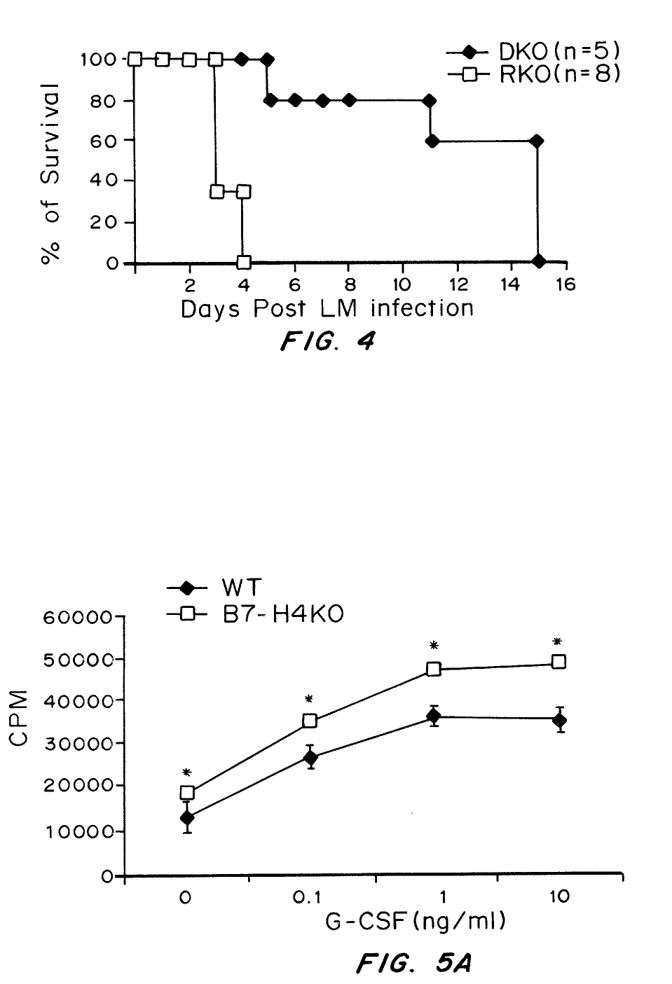

RKO and DKO mice were then challenged by administration of a lethal dose of LM to examine their innate resistance. Infection of RKO mice by LM led to exponential growth of LM in liver and 100% mortality by day 4 (FIG. 4). In sharp contrast, DKO mice have significantly less bacterial load in the liver at day 2 and the majority of the mice were able to survive more than 10 days LM challenge (FIG. 4). Similar exponential growth of LM in other organs including spleens were observed, indicating a dissemination of LM infection. In contrast to long-term survival of a significant fraction of infected 36 background 37-H4KO mice (FIG. 2a), all DKO mice eventually died of infection at day 15, supporting an important role of adaptive immunity (FIG. 4). Combined with rapid clearance of LM from liver and other organs in DKO mice as early as day 2, the results indicate that lack of B7-H4 confers enhanced innate immunity against LM infection, which is largely mediated through increased granulocytes.

### Example 4

### B7-H4 Directly Inhibits Proliferation of Granulocytes

Bone marrow cell culture and granulocyte growth and inhibition assay Bone marrow cells were aspirated and prepared as described by Wilcox, R. A. et al., Blood, 103:177-184 (2004). For B7-H4-mediated growth inhibition, B7-H4Ig or control murine Ig were coated in 96-well plates overnight. After extensive washing, BM cells were plated 2×10⁶/well in 24-well plates with or without recombinant murine G-CSF (Pepro Tech Inc., Rocky Hill, N.J.) at indicated concentrations. Cells were harvested at indicated time points and cell numbers were counted with Beckman Coulter Counter (Beckman, Fullerton, Calif.). To examine cell growth, 2×10⁵/well of BM cells were plated in 96-well plates with G-CSF. After being pulsed with ³HTdR, cells were harvested with FilterMate® cell harvester (Perkin Elmer, Shelton, Conn.) 16 hours post ³HTdR pulse. The incorporated ³HTdR was detected by Trilux® Liquid Scintillation and Luminescence Counter (Wallac, Turku, Finland). For cell division assay, BM cells were first labeled with 2 μM of carboxfluorescein diacetate succinimidyl ester (CFSE, Invitrogen, Carlsbad, Calif.) and then were added to the cultured in 96- or 24-well plates. Cells were harvested at indicated time points, stained with mAb Gr-1 and CD11b and subjected to flow cytometry analysis for CFSE content (2) at different time points.

Results

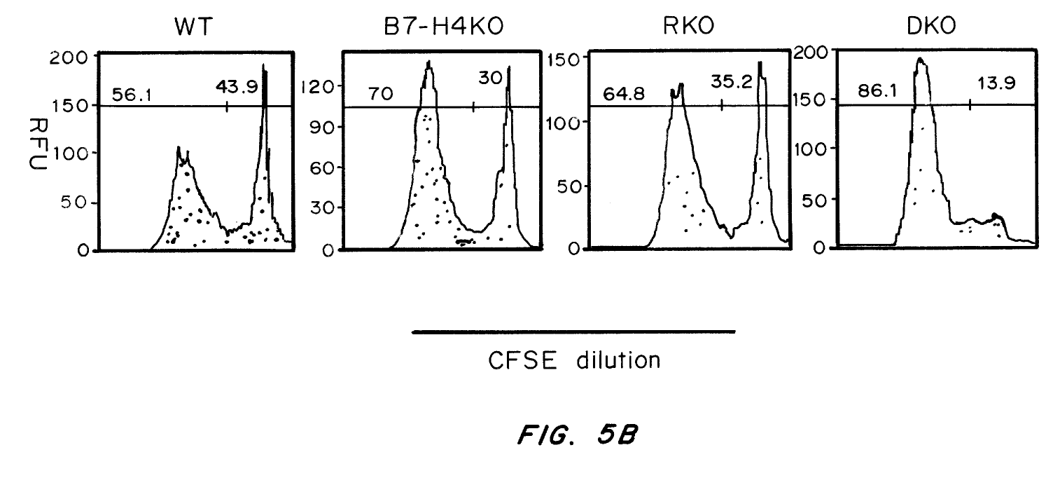

Increased granulocytes in B7-H4KO mice suggest that B7-H4 play a role in delivering an inhibitory growth signal to granulocytes. Granulocytes from 37-H4 KO mice were examined to determine whether they have better growth potential than WT granulocytes. To do so, bone marrow (BM) cells, which contain large numbers of granulocyte precursors, were prepared and cultured from WT or B7-H4 KO mice in the presence or absence of G-CSF for 3 days to facilitate differentiation of granulocyte/neutrophil. The proliferation of BM cells was subsequently determined by ³HTdR incorporation. FIG. 5A shows that while BM cells respond to G-CSF by proliferating in a dose-dependent fashion, proliferation of BM cells from B7-H4KO mice was significantly higher than those from WT mice. Flow cytometry analysis of BM cells which respond to G-CSF in the end of culture, shows that more than 95% of survived cells are CD11b+Gr-1+ granulocytes. While this data is consistent with an inhibitory effect of B7-H4 in granulocytes, other cellular components in BM cells may also contribute to proliferation. To precisely exclude this possibility, BM cells were labeled with CFSE and after stimulation with G-CSF for 3 days, the cells were stained with anti-Gr-1+/CD11b+ mAbs to monitor granulocytes for cell division. FIG. 5b shows that 70% Gr-1+CD11b+ granulocytes from B7-H4KO mice (B6) divide at least once whereas only 56% granulocytes from WT B6 mice had diluted CSFE. Similar, but more significant differences were found in mice with the RAG-1 KO background: 86% granulocytes from DKO mice entered division whereas only 64.8% granulocytes from RKO mice had diluted CSFE. The results thus indicate that lack of B7-H4 on BM cells increase proliferation of BM-derived granulocytes.

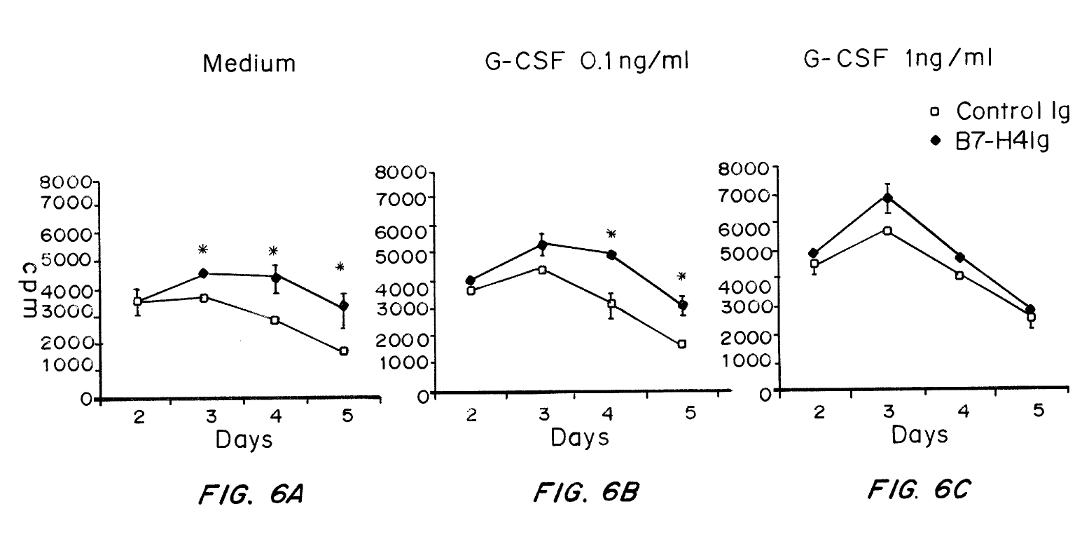

Considering that the lack of B7-H4 could result in increased proliferation of BM-derived granulocytes, whether B7-H4 could directly inhibit their proliferation was determined. To test this, WT BM-derived granulocytes were cultured in the presence of recombinant B7-H4Ig fusion protein and examined proliferation of granulocytes. Proliferation of WT BM cells was significantly inhibited by B7-H4Ig, a fusion protein of B7-H4 extracellular portion and immunoglobulin Fc. The inhibition was evident at day 3 of the culture and became more significant at day 4 and 5 (FIG. 6a). Addition of 0.1 ng/ml of G-CSF in the culture, albeit moderately increasing proliferation of BM cells, did not significantly overcome B7-H4Ig mediated suppression (FIG. 6b). Increasing G-CSF to 1 ng/ml in the culture, however, could recover B7-H4Ig-mediated growth inhibition of BM cells in large degree (FIG. 6c). Similar inhibition was also observed in B7-H4 deficient granulocytes. Combined together, the results provide further evidence that B7-H4 is inhibitory for the proliferation of granulocytes, which could be reversed by G-CSF.

It has been discovered that B7-H4 can negatively regulate innate immunity against Listeria infection. It is believed that the effect of B7-H4 is mediated through growth suppression of granulocytes. In the context of broad expression pattern of B7-H4 in peripheral tissue, the data supports B7-H4 as an important regulatory molecule in the control of innate immunity in peripheral tissues, in addition to the previously described role of B7-H4 in the inhibition of T cell responses.

In B7-H4KO mice, the majority of the extracellular portion of B7-H4 protein is deleted to assure complete elimination of interaction between endogenous B7-H4 and its putative receptor. Ablation of this gene, however, does not have a profound effect on T cell responses to polyclonal and allogeneic antigen stimulation in vitro. Similar observations have been made in a recent study reported by Suh, W. K. et al. Mol. Cell. Biol., 26:6403-6411 (2006). While these findings indicate that B7-H4 does not substantially influence the inhibition of strong polyclonal T cell responses to CD3 cross-linking or allogeneic antigens, it is possible that B7-H4 affects more selective steps during cascade of T cell responses. For example, a recent study shows that although B7-H4KO mice responded normally to several types of airway inflammatory responses as well as LCMV and influenza infection, the mice have slightly enhanced T-cell immune responses to Leishmania major infection. Responses of granulocytes in this knock-out system, however, were not examined. The experiments indicate that a dominant role of B7-H4 in Listeria infection is to suppress granulocyte-mediated innate immunity and this effect could also be observed in RAG-1 KO mice in the absence of adaptive immune system. Therefore, in addition to inhibition of T cell immunity as reported previously, B7-H4 may play a critical role in negative regulation of innate immunity against bacterial infection.

Although there is slightly increased granulocytes in the spleens of B61B7-H4KO mice, dramatic increase of granulocytes occur upon LM infection (FIG. 2). This increase, however, is not simply due to increased recruitment by LM-induced inflammation. B7-H4 KO mice in B6 background have a small increase of granulocytes in blood, bone marrow and spleen without infection. A more dramatic elevation of granulocytes is observed in RAG-1 KO background. In addition, bone marrow cells from B7-H4KO mice produce more granulocytes in the presence of G-C SF stimulation. Finally, inclusion of B7-H4 protein in culture significantly inhibits growth of bone marrow-derived granulocytes. The role of B7-H4 in the inhibition of granulocytes could be reversed, at least partially, by addition of higher concentrations of G-CSF in culture. G-CSF is a critical factor for growth and homeostasis of granulocyte in vivo. The result suggests that B7-H4 may serve as a negative regulator to antagonize the role of G-CSF in vivo. Combined together, the results support that B7-H4 provides an inhibitory signal for responsiveness of granulocytes to G-CSF, a foremost growth factor for granulocytes, and thus may regulate homeostasis of granulocytes.

It has been shown that B7-H4, upon binding to its putative receptor, inhibits cell cycle progression on T cells (Sica, G. L. et al., Immunity 18:849-861 (2003); Kryczek, I. et al. J Eicp Med, 203:871-881 (2006)). In the cell culture system, dilution of CFSE and incorporation of ³HTdR are clearly inhibited (FIG. 6a). Bone marrow cells were observed to undergo proliferation (FIG. 6a) and cell division (FIG. 5g) in the absence of exogenously supplied G-CSF, a key growth factor for granulocytes. It is possible that endogenous G-CSF is produced by bone marrow cells and maintains basal level of proliferation in vitro. This suppression could be largely reversed by adding G-CSF (FIG. 6c). During the culture, significant increases of cell apoptosis was not observed for up to 5 days. Therefore, growth inhibition may be a dominant mechanism in granulocytes by B7-H4 ligation. B7-H4 mRNA is widely expressed by various cells while its cell surface expression could be largely contained in cytoplasm as observed in ovarian cancer and infiltrating macrophages (Kryczek, I. et al., J Eicp Med, 203:871-881 (2006)). Surface expression of B7-H4 could be regulated by cytokines within the bone marrow microenvironment to inhibit granulocyte growth.

Granulocytes, including neutrophils, are one of the earliest cells to arrive at the site of an infection and are the first line of individual defense against infection through their capacity to phagocytose (Nathan, C. Nature Rev. Immunol., 6:173-182 (2006)). The findings showing an increased resistance to Listeria infection in B7-H4KO mice implicates a new approach to enhance innate immunity against infection by Listeria and possibly other pathogens. It is also interesting that B7′-H4 KO mice in the RAG-1 background have a more profound increase in the number of granulocytes and are more resistant to early phase LM infection in comparison with B7-H4 KO mice in B6 background. These data implicate a possible suppressive role of adaptive immunity components including T and B cells in granulocyte homeostasis and response to Listeria infection. Therefore, the method to selective blockade of B7-H4 expression such as neutralizing mAb or appropriately engineered B7-H4 protein with antagonistic activity represents a new approach to increase granulocytes and enhanced innate immunity against pathogen infection.

### Example 5

### Soluble B7-H4 in the Sera of Rheumatoid Arthritis Patients Correlates with Disease Severity

Patients and Healthy Donors:

Sera samples were obtained from 68 patients with diagnosed RA, 35 patients with diagnosed SLE and 24 normal healthy donors under approval of the Internal Review Board of Mayo Clinic. RA patients were classified to 4 groups as follows. 0: no active disease, 1: 1-4 active joints, 2: 5-9 active joints, 3: more than 10 active joints with or without extraarticular disease.

Detection of Soluble B7-H4, Collagen-Specific Autoantibodies and Anti-dsDNA Autoantibody:

For detection of human sH4, specific mAb hH4.3 (2 μg/ml) and hH4.1 (2 μg/ml) against human B7-H4 was used as capture and detection, respectively, in ELISA. To remove Rheumatoid Factor, the sera were treated with human IgG agarose (Sigma-Aldrich, St. Louis, Mo.) before detection in ELISA. For measurement of collagen-specific autoantibodies, chicken collagen (1 μg/ml) was coated on the plate overnight at 4° C., and biotin conjugated anti-mouse IgG, IgG1, IgG2a and IgG2b Ab (BD, San Jose, Calif.) as detection antibodies. To measure anti-dsDNA autoantibody levels, dsDNA from salmon testes at 10 μg/ml in PBS was coated on the plate overnight at 4° C., and HRP conjugated anti-mouse IgG, (BD San Jose, Calif.).

Western Blot:

The sera was mixed with 2× sample buffer (4% SDS, 0.2% bromophenol blue, 20% glycerol in 100 mM Tris buffered saline) and boiled for 5 min. The samples were electrophoresed under reducing conditions on a 10% Ready gel (Bio-Rad, Richmond, Calif.) and the proteins electroblotted onto Protran BA85 (Whatman, Florham Park, N.J.). The Immobilon-P sheet was blocked in 5% nonfat dry milk in PBS for 1 h and incubated with the antibody at 4° C. overnight. After repeated washing (five times 5 min), bound antibody was detected with horseradish peroxidase (HRP)-labeled.

Results

To detect sH4, sera from individual patients with diagnosis of rheumatoid arthritis based on American Rheumatism Association criteria were analyzed by enzyme-linked immunosorbent assays (ELISA) using two specific monoclonal antibodies (mAb) binding to different epitopes on human B7-H4. In this assay, 65% (44 out of 68) samples from patients with RA and 43% (15/35) from patients with SLE were above background and therefore positive. Evaluation of sH4 in healthy donors (HD) showed only 13% (3/24) were positive (FIG. 7a). sH4 is significantly higher in RA and SLE patients than healthy donors (P<0.05). In addition, the mean concentration of sH4 in RA (96.1 ng/ml) and SLE (36.9 ng/ml) was significantly higher than those of the healthy donors (3.8 ng/ml). The results indicate that sH4 is elevated in a significant portion of RA and SLE patients.

Western blot analysis was used to validate the presence of sH4 in sera from 3 patients with rheumatoid arthritis. Using specific mAb against B7-H4, the sera from 3 RA patients, who have detectable sH4 in ELISA, showed a single 50-kDa band. This matched the size of predicted extracellular domain of human B7-H4. In contrast, no band was observed in sera from three healthy donors (FIG. 7b). The data support the presence of sH4 in the sera of RA patients.

The association of elevated concentration of sH4 with the severity of RA was investigated. Based on severity of diseases, 68 RA patients were classified into 4 groups (0-3) with most severe diseases in group 3 as described in Methods. The mean concentration of sH4 in group 3 (260.7 ng/ml) was significantly higher than those of group 0 (22.0 ng/ml) or Group 1 (18.8 ng/ml). However, there was no significant difference among group 0-2 by Scheffe test (FIG. 7c). The data thus indicate that RA patients in group 3 have highest level sH4 and suggest that sH4 might play a role in the progression of severe RA.

### Example 6

### Soluble B7-H4 Exacerbates Collagen-Induced Arthritis in a Mouse Model

Mice

Male DBA/1j mice, MRL-lpr/lpr mice and C57BL/6-lpr/lpr (B6-lpr/lpr) were obtained from the Jackson Laboratory (Bar Harbor, Me.). Age-matched mice, 4-10 weeks old, were used for all experiments. B7-H4KO mice were generated in this laboratory as described above and have been backcrossed to B6 background for 10 generation. DBA/1j×B7-H4KO mice were generated by backcrossed B7-H4KO mice into DBA/1j backgrounds for 5 generations. B6-lpr/lpr×B7-H4KO mice were obtained by backcrossing between B6-lpr/lpr and B7-H4KO mice. All mice were maintained in the Animal Facility at Johns Hopkins Hospital under approval protocol by the Institutional Animal Care and Use Committee.

Induction of Collage-Induced Arthritis:

CIA was induced in 8-10 weeks old male DBA/1j mice by intradermal tail base injection of 0.2 mg chicken collagen (Sigma-Aldrich, St. Louis, Mo.) in 0.05 M acetate acid, supplemented with 4.0 mg/ml mycobacterium tuberculosis (DIFCO, Detroit, Mich.) emulsified in complete Freund adjuvant. Fourteen days after first primary immunization, the mice were identically boosted once. Severity of disease was evaluated by visual inspection of the paws. Each paw was scored for the degree of inflammation on a scale from 0 to 4: 0, no evidence of erythema and swelling; 1, erythema and mild swelling confined to the midfoot (tarsals) or ankle joint; 2, erythema and mild swelling extending from ankle to the midfoot; 3, erythema and mild swelling extending from ankle to metatarsal joints; 4, ecrythema and severe swelling encompassing the ankle, foot and digits. Scores from all four paws were added to give the total for each animal.

Murine B7-H4 Constructs

B7-H4Ig construct was prepared as described by Sica, G. L. et al. B7-H4, a molecule of the B7 family, negatively regulates T cell immunity. Immunity 18, 849-61 (2003)). To generate B7-H4V and B7-H4VC plasmids, 2 flanking 5′ and 3′ primers were designed with XhoI and EcoRI restriction sites, respectively (5′primer; 5′-ccgctcgagccaccatggcttccttggggcag-3′ (SEQ ID NO:6), 3′primer for B7-H4V; 5′-cggaattccgctaatttatctctggcatact-3′ (SEQ ID NO:7), 3′primer for B7-H4VC; 5′-cggaattccgctaagagttcagcaactgcag-3′ (SEQ ID NO:8)). Appropriate regions of cDNA were amplified using primers. PCR product was digested with XhoI and EcroRI and ligated into XhoI/EcroRI-digested pcDNA3.1 vectors (Invitrogen, Carlsbad, Calif.).

Collagen-Specific T Cell Proliferation and Cytokine Production.

The spleen was removed on day 14 after the last immunization. CD4+ T cells were purified by using magnetic beads (Miltenyi Biotec, Auburn, Calif.). Whole splenocytes or purified CD4+ T cells were stimulated with denatured (60° C., 30 min) chicken type II collagen (CII) in 96 well flat bottom microtiter plates for 72 hr, and pulsed with [³H] thymidine (1 μCi/well) (Amersham Pharmacia Biotech, Piscataway, N.J.) for the last 12 hr. In the culture of purified CD4+ T cells, irradiated (50Gy) splenocytes from the syngeneic mice were added as antigen-presenting cells. Supernatants from the cultures were collected after 48 hr and assayed for mouse IFN-γ (BD, San Jose, Calif.) and IL-17A (eBioscience) using ELISA kit according to the protocols recommended by manufacturer.

Results

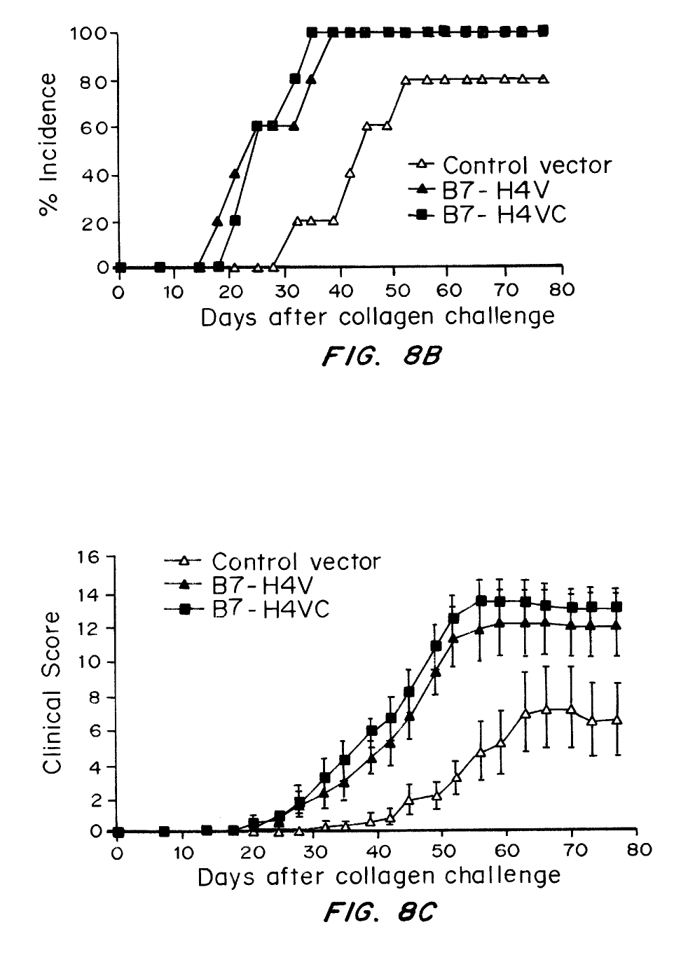

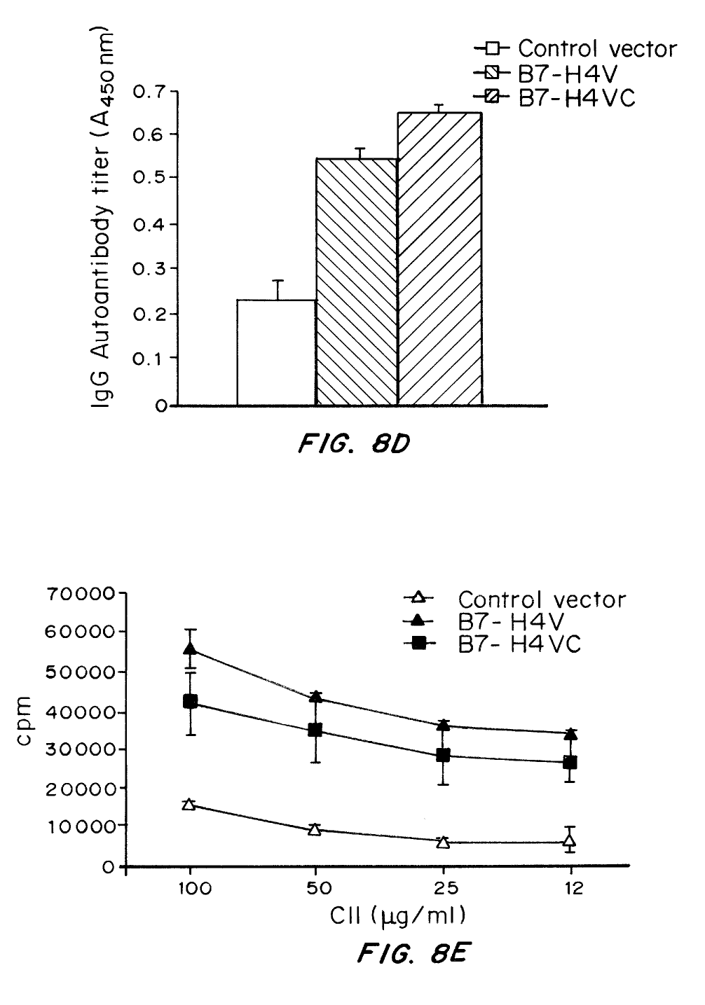

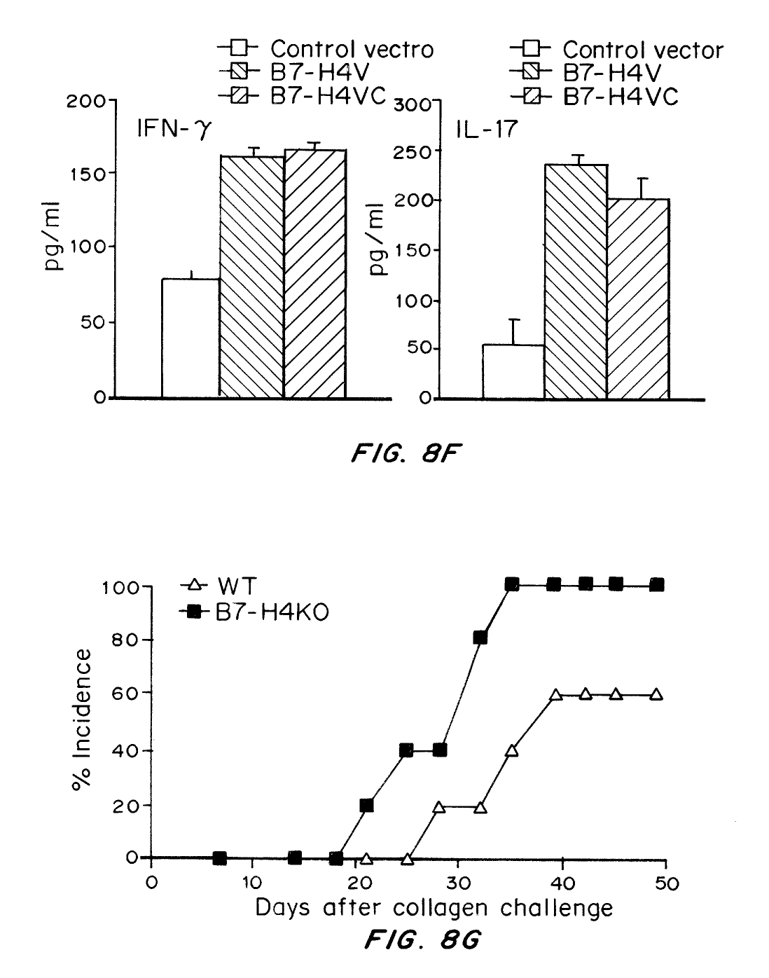

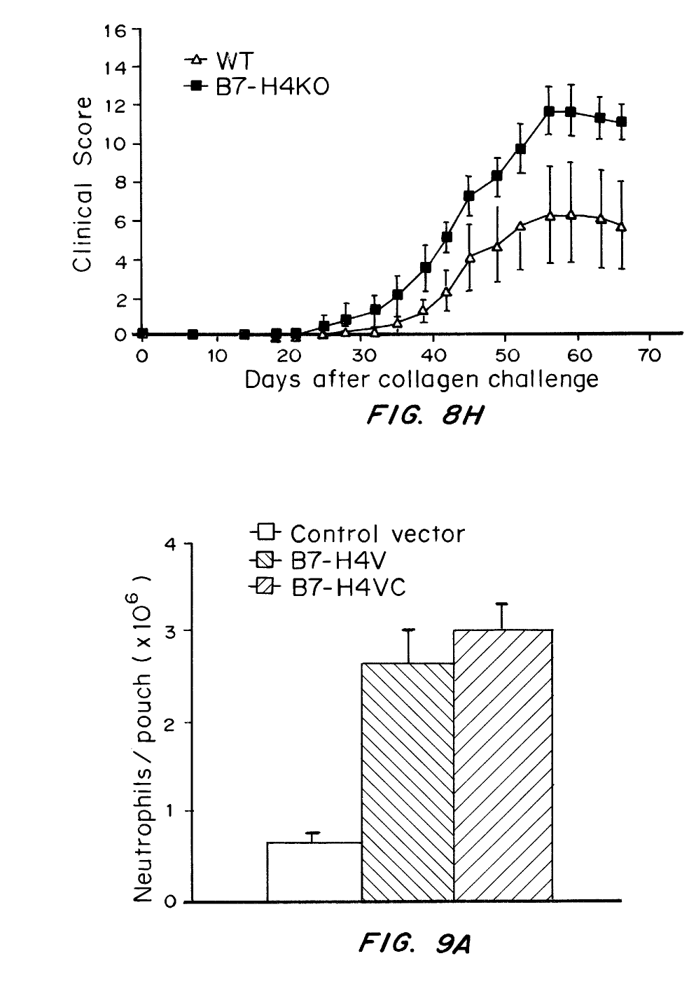

To recapitulate and explore possible role of sH4 in the pathogenesis of RA, a mouse model of collagen-induced arthritis (CIA) was used. CIA is a well-characterized mouse model for human arthritis, in which injection of collagen into DBA/1j mice induces swelling and progressive inflammation in large joints and lead to arthritis. To express sH4 in vivo, an expression vector, B7-H4VC, was constructed in which the transmembrane and intracellular domains of mouse B7-H4 cDNA were deleted, and the truncated gene encoding both IgV and IgC domains were placed under the control of CMV immediate early promoter. Another vector, B7-H4V, containing only IgV domain of B7-H4 was also produced (FIG. 8a). Upon expression, these truncated proteins/polypeptides are expected to compete with endogenous B7-H4 on the cell surface to bind its putative receptor. Parental vector is included as the control. By a hydrodynamic expression procedure known in the art, injection of these plasmids led to expression of sH4 up to 2 μg/ml in the sera, based on specific capture sandwich ELISA using two anti-murine B7-H4 mAb.

In the CIA model, immunization of DBA/1j mice with collagen led to appearance of arthritic symptom starting around 28 days. Control vector-treated mice developed arthritis beginning at day 32 and 80% of mice developed disease on day 60 after first immunization. Injection of B7-H4VC led to earlier development of disease (17 days) and 100% mice developed arthritis around 30 days. Similar results were also seen in the mice injected with B7-H4V (FIG. 8b). Furthermore, treatment by either B7-H4V or B7-H4VC significantly increase severity of arthritis as indicated by increased clinical score (FIG. 8c), increased swelling of footpad and increased infiltration of inflammatory cells in joints as shown in histopathology analysis.

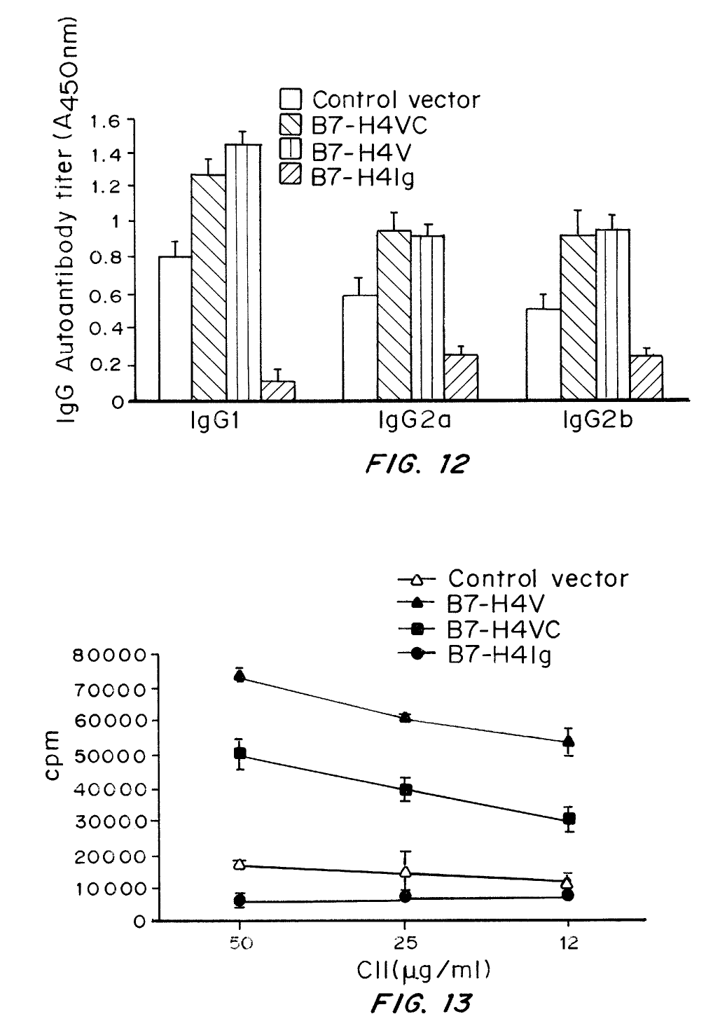

Assessment of cellular and humoral immune responses revealed that increased incidence and severity of arthritis was accompanied with elevated total IgG autoantibodies (FIG. 8d) as well as other subtypes including IgG₁, IgG₂ₐ and IgG_{2b} to collagen CII at day 30 after immunization and B7-H4VC or B7-H4V treatment (FIG. 12). Stimulation of total spleen cells or purified CD4+ T cells from mice, which were treated with B7-H4VC or B7-H4V, by CII also induced much higher level of proliferation in comparison with mice treated with control vector (FIG. 8e and FIG. 13). Importantly, IFN-γ and IL-17, two major cytokines responsible for CIA progression, also increase significantly in the cultures (FIG. 8f). Taken together, the data demonstrate that sH4 enhance autoimmune responses against CII and exacerbate autoimmune CIA.

If B7-H4VC and B7-H4V act as a decoy to block the effect of endogenous B7-H4 on the cell surface, a similar exacerbation effect that should also be observed in B7-H4 deficient mice (B7-H4KO). To test this, B7-H4KO phenotype mice were backcrossed to DBA/1j background for 5 generations. B7-H4KO-DBA/1j mice develop normally and do not have obvious abnormality in gross appearance and development of immune system. These mice, however, developed much more severe CIA, showing higher accidence (FIG. 8g) and clinical score (FIG. 8h) than B7-H4+/+control mice, results similar to those from B7-H4VC or B7-H4V-treated mice. Therefore, the data support that sH4 functions as a decoy molecule to increase autoimmune responses and exacerbate CIA.

### Example 7

### Increased Neutrophils are Responsible for Exacerbation of CIA by sH4

Air Pouch Assay for Neutrophils

The air pouch assay was performed as described by Edwards, J. C. et al., J Pathol, 134-147-56 (1981). Briefly, mice were anesthetized with 2,2,2-Tribromoethanol (Sigma-Aldrich, St. Louis, Mo.) and subcutaneous dorsal pouches were created by injection of 5 ml of sterile air. After 3 day, pouches were re-injected with 3 ml air. On day 6 after the first injection, 50 μg LPS in 1 ml PBS was injected into the pouches. Five hours later, mice were anesthetized and pouches were lavaged with 3 ml PBS to collect infiltrating cells.

Results

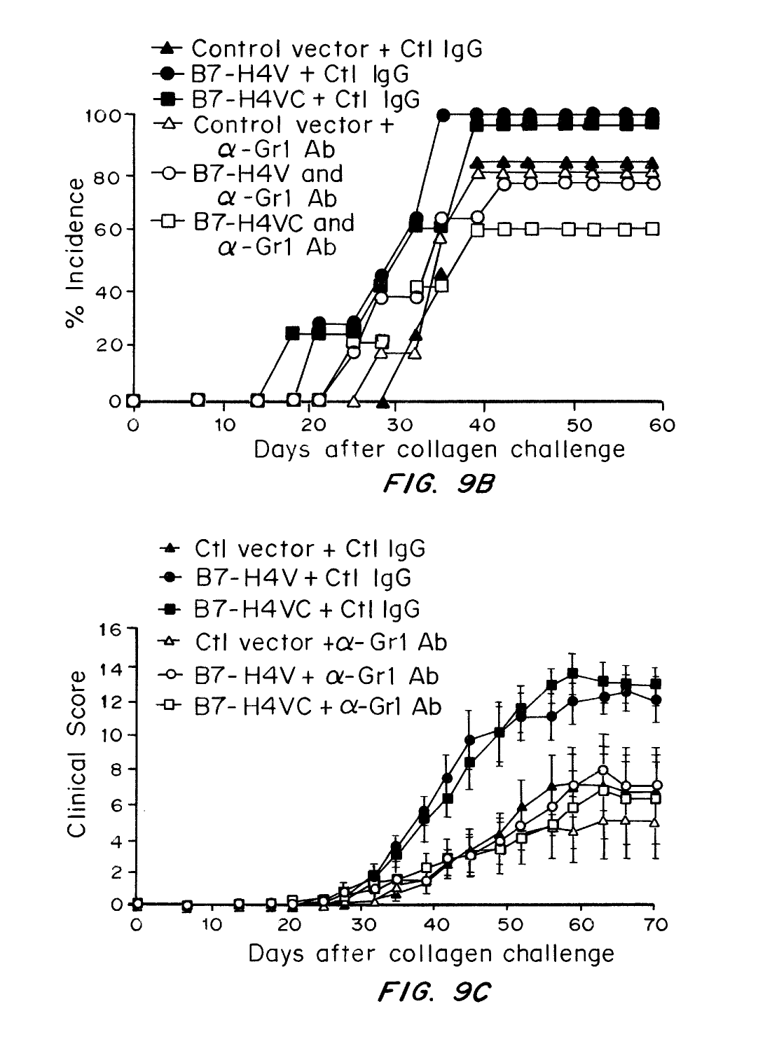

B7-H4KO mice are resistant to Listeria infection due to rapid increase of neutrophils. Further experiments demonstrated that B7-H4 could directly inhibit growth of neutrophil progenitors. Therefore, sH4 may block endogenous B7-H4 and thereby exacerbate CIA via neutrophil-mediated inflammation, a hypothesis which may provide an interpretation for progressive inflammation of RA. Whether or not expression of sH4 increases neutrophils in murine peripheral tissues was explored. Due to difficulty to directly access neutrophil number in RA lesions in mouse, an air pouch assay in which neutrophils could be collected from subcutaneous air pouches upon induction of inflammation were used. As shown in FIG. 9a, mice injected with B7-H4V or B7-H4VC had significantly more neutrophils in each air pouch than that of control vector. Together with previous studies in B7-H4KO mice, the results indicate that sH4 induce a rapid increase of neutrophils in peripheral tissues in vivo.

Neutrophils were depleted to investigate whether the effect of sH4 in CIA exacerbation could be eliminated. CIA-mice were treated with B7-H4VC or B7-H4V and subsequently treated with anti-Gr-1 antibody every other day to deplete neutrophils. Enhanced effect of B7-H4V or B7-H4VC in both CIA incidence (FIG. 9b) and clinical score (FIG. 9c) was completely eliminated by anti-Gr-1 antibody treatment. The results thus support that neutrophils are responsible for the effect of sH4 in the progression of CIA.

### Example 8

### Soluble B7-H4 Exacerbates SLE-Like Diseases in Lpr Mice and Enhances Autoimmune Responses

Urine Protein Excretion

Urinary protein excretion was determined by dipstick analysis (GERMAINE, San Antonio, Tex.). The proteinuria grade was scored from 0 to 4 as follows: grade 0, normal; grade 1, 30 mg/dl; grade 2, 100 mg/dl; grade 3, 300 mg/dl; grade 4, 2000 mg/dl.

Histological Assessments of Arthritis and Nephritis

CIA mice were sacrificed at day 35. The hind paws were removed, fixed in Formalin, decalcified in 10% EDTA, embedded in paraffin, sectioned, and stained with H&E. For histological evaluation of renal disease, mice were sacrificed at 6 months of age. Kidneys were either fixed in formalin or snap-frozen in Tissue Tek (Sakura Finetek, Torrance, Calif.) for cryostat sectioning. Formalin-fixed tissue was embedded in paraffin, sectioned, and stained by the periodic acid-Schiff (PAS) method. Frozen sections were fixed in acetone and 1% paraformaldehyde, and stained with FITC-conjugated anti-mouse IgG Ab or C3 Ab (ICN/Cappel, Aurora, Ohio).

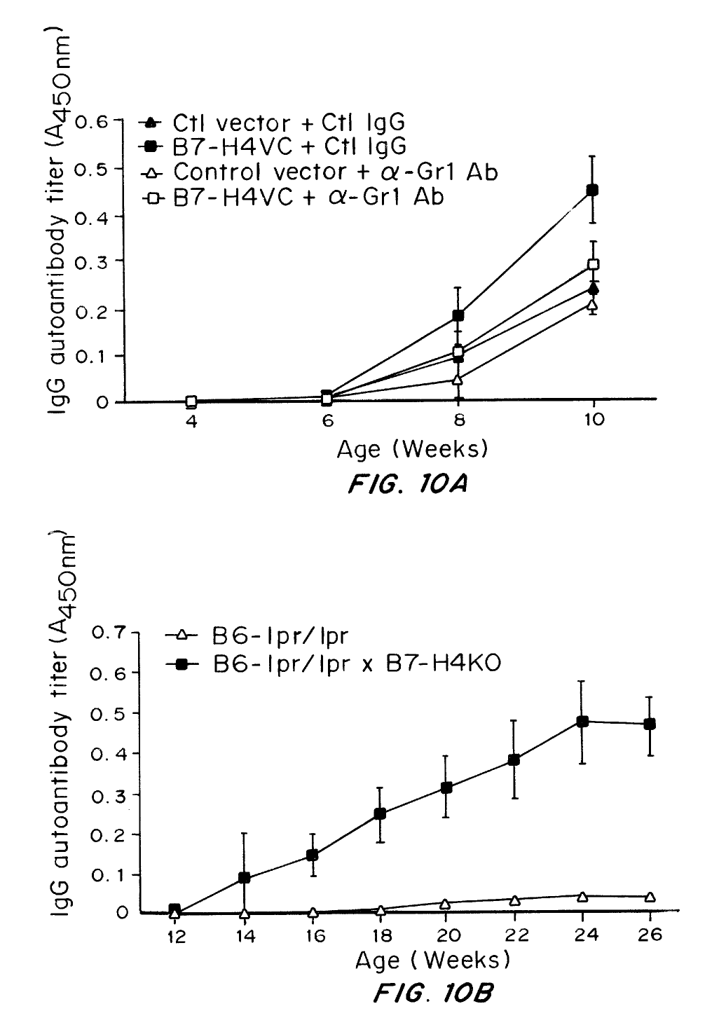

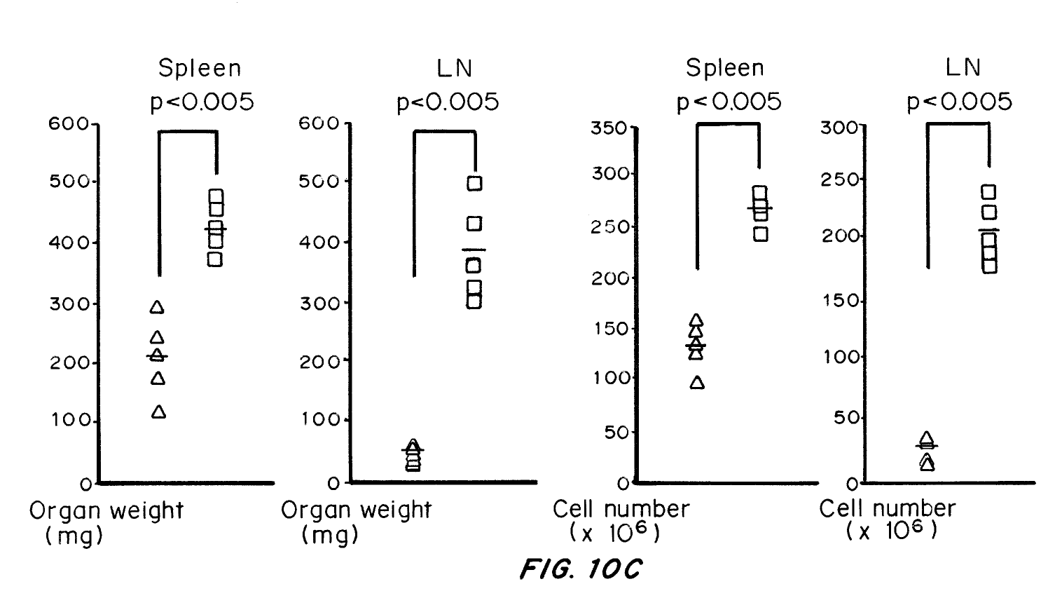

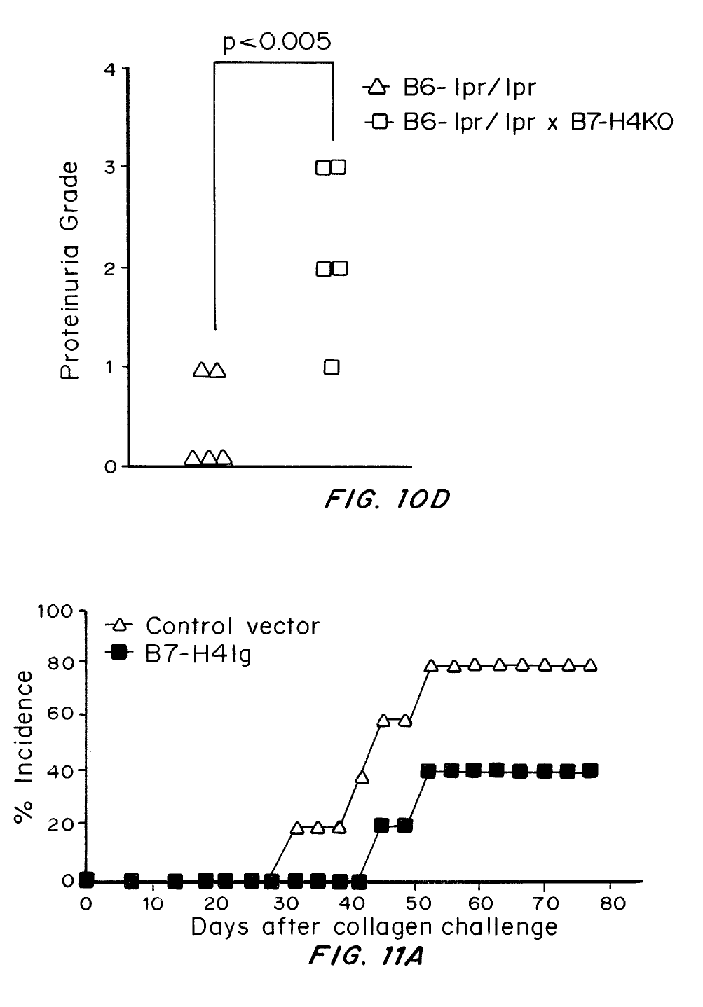

A significant fraction of SLE patients also have detectable sH4 in sera (FIG. 7a). It is possible that sH4 may also play a role in the progression of SLE. To test this, sH4 was investigated to determine whether it could promote autoimmunity in MRL-lpr/lpr mice, in which the mice spontaneously develop progressive SLE-like symptoms largely due to the effects of autoantibodies and lymphoproliferation. MRL-lpr/lpr mice were treated with the B7-H4VC plasmid and anti-dsDNA autoantibodies in sera were evaluated. As shown in FIG. 10a, upon treatment by the B7-H4VC, concentration of anti-dsDNA autoantibodies in sera elevated significantly higher than the mice treated with control plasmid at 10 weeks. Depletion of neutrophils by injection of anti-Gr-1 antibody completely eliminated this effect, a result similar to the observation in the CIA model. This initial study suggests that sH4 also plays a role in promoting autoimmune responses in this SLE model.

To facilitate analysis of the immune responses and the role of sH4 in the pathogenesis of SLE, B7-H4−/− phenotype mice were backcrossed to 136-lpr/lpr mice, a strain with similar but less aggressive SLE-like symptoms as the MRL-lpr strain. As expected, anti-dsDNA IgG autoantibodies were developed much earlier and in much higher titers in B6-lpr/lpr×B7-H4KO mice than the control B6-lpr/lpr mice (FIG. 10b). Importantly, B6-lpr/lpr×B7-H4KO mice rapidly developed severe splenomegaly and lymphoadenopathy with significantly increased weight (FIG. 10c) compared with control B6-lpr/lpr mice. The spleen and lymph nodes were much larger and cellularity of these organs increased significantly in B6-lpr/lpr×B7-H4KO mice than the controls (FIG. 10c). The major cell components, which are increased significantly upon sH4 treatment in these organs, are neutrophils (Gr-1+CD11b+) and T cells (CD3+CD8+, CD3+CD4+ and CD3+CD4−CD8−B220+). B6-lpr/lpr×B7-H4KO mice developed severe glomerulonephritis with interstitial inflammatory cells infiltrates, hypercellular glomerulus and increased mesangial cells. In addition, the mice also developed vasculitis with perivascular cell infiltration, the glomerular deposition of total IgG) and C3 as well as increased proteinuria (FIG. 10d) within 30 weeks. In contrast, control B6-lpr/lpr mice have normal kidneys without any visible pathology up to 24 months. Taken together, the results demonstrate that sH4 exacerbates SLE-like diseases in lpr mice by enhancing antibody and cell-mediated autoimmune responses and pathology.

### Example 9

### Inhibition of CIA Progression by B7-H4Ig

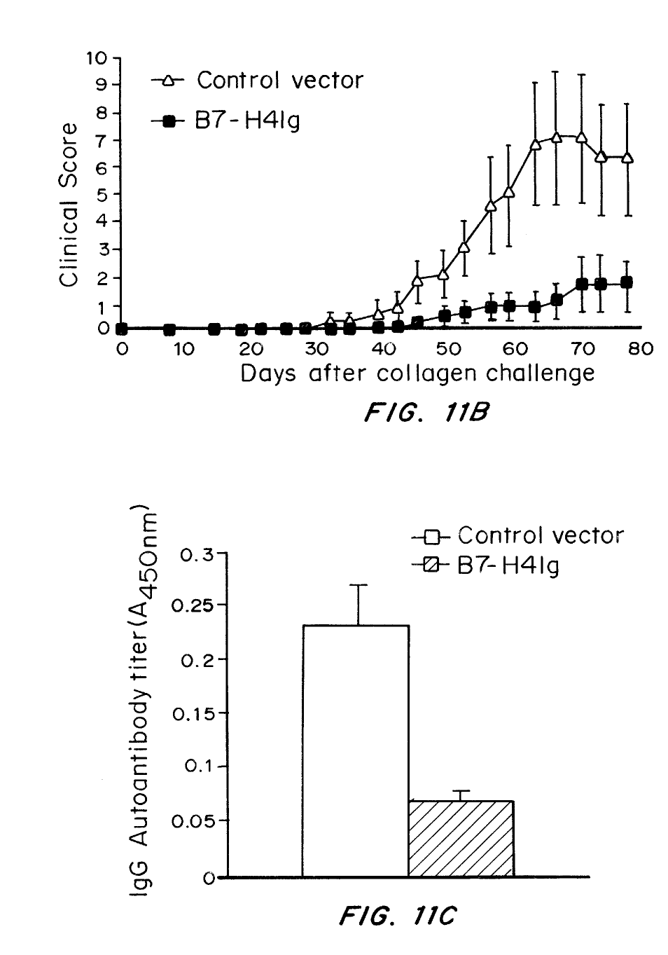

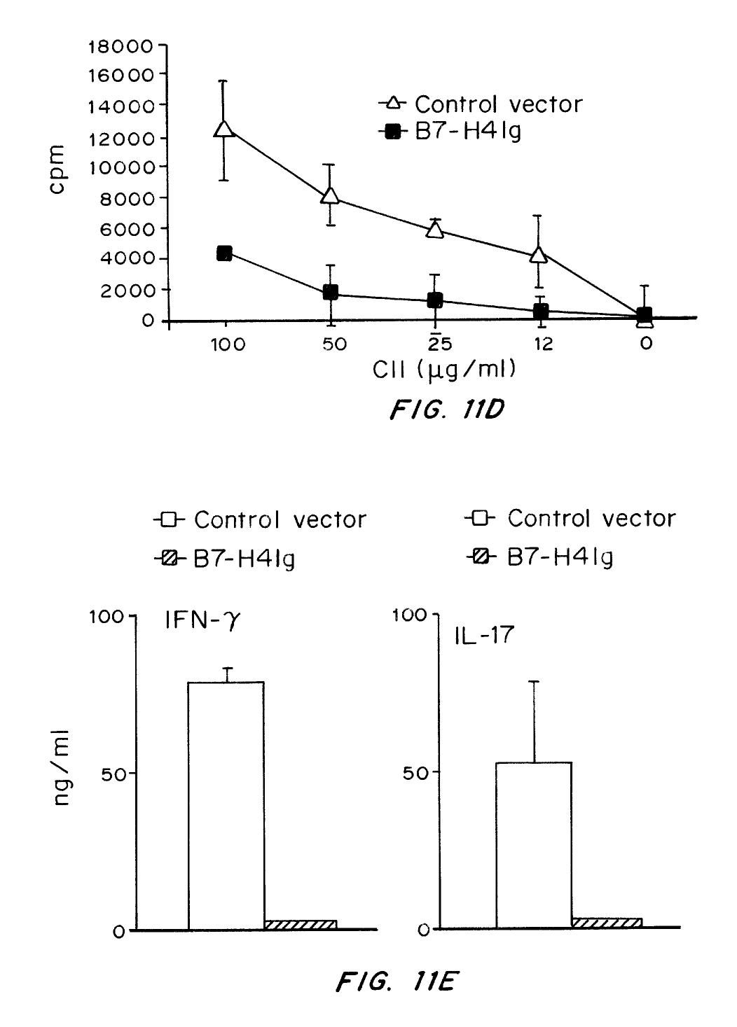

While the data show that sH4 in RA and SLE murine models promotes progression of diseases, these data also support that endogenous B7-H4 is a checkpoint molecule in suppressing autoimmune responses. Therefore, a potential approach to suppress these autoimmune diseases is to increase the expression of B7-H4 in agonist form in order to engage its putative receptor. The effect of B7-H4Ig fusion protein in which B7-H4 extracellular domain was fused to murine IgG2a Fc portion was described by Sica, G. L. et al. B7-H4, a molecule of the B7 family, negatively regulates T cell immunity. Immunity 18, 849-61 (2003); Chapoval, A. I., Zhu, O. & Chen, L. Immunoglobulin fusion proteins as a tool for evaluation of T-cell costimulatory molecules. Mol Biotechnol 21, 259-64 (2002). The Fc portion of B7-H4Ig could bind the Fc receptor to facilitate an agonist effect in vivo. The effect of B7-H4Ig in the progression of CIA was then tested. In comparison with control plasmid, B7-H4Ig plasmid treatment one day before CII challenge significantly decreased arthritis incidence and clinical score, as well as delayed the onset of CIA (FIG. 11a & b). Furthermore, B7-H4Ig plasmid treatment suppressed the production of total IgG (FIG. 11c) and IgG₁, IgG₂ₐ and IgG_{2b} autoantibodies to CII (FIG. 12). Proliferation of splenocytes and CD4+ T cells (FIG. 11d and FIG. 13) as well as IFN-γ and IL-17 production in response to CII were also significantly suppressed upon B7-H4Ig treatment (FIG. 11e). Collectively, the results demonstrate that B7-H4Ig could work as an agonist to suppress both humoral and cellular autoimmunity. In addition, this method should also be effective in suppressing pathogenesis of CIA.

### Example 10

### Expression of B7-H4Ig in MRL-lpr/lpr Mice Increases Survival

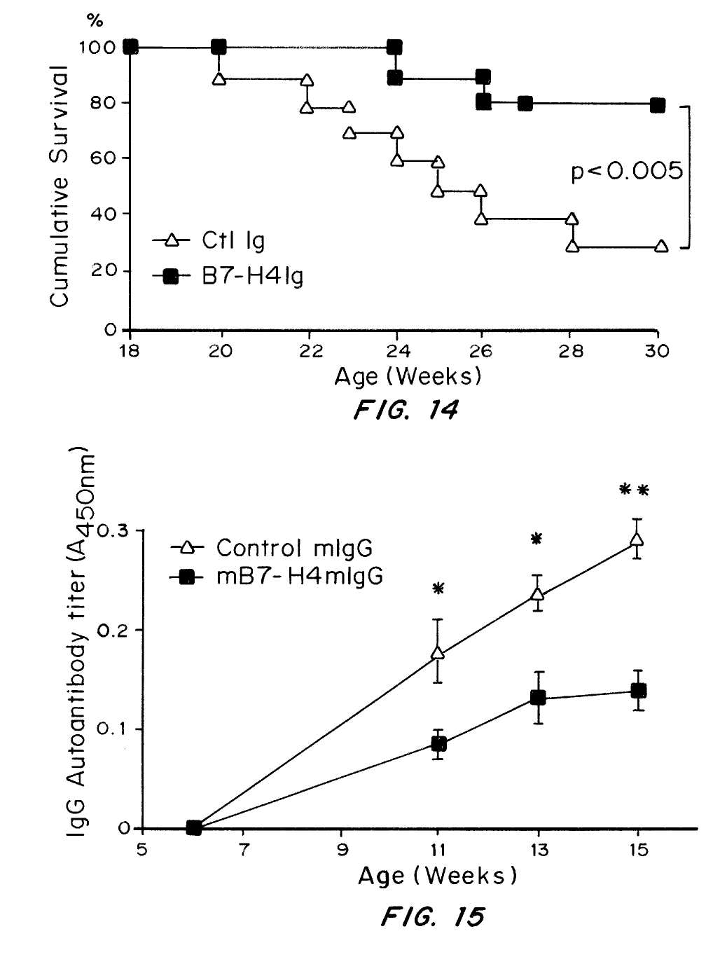

MRL-lpr/lpr mice were injected with control mIgG plasmid or B7-H4 μg plasmid at 6, 8, 10 and 12 weeks of age. All phenotypes were analyzed at 19 weeks of age. Each group contained 5-10 mice and each set of experiments were repeated at least twice. FIG. 14 is a line graph of percent cumulative survival versus age (weeks) in MRL-lpr/lpr mice injected with control mIgG plasmid (□) or B7-H4Ig plasmid (▪) at 6, 8, 10 and 12 weeks of age. FIG. 14 shows that treatment by B7-H4Ig (murine) vector increases survival of MRL-lpr/lpr mice. All phenotypes were analyzed at 19 weeks of age.

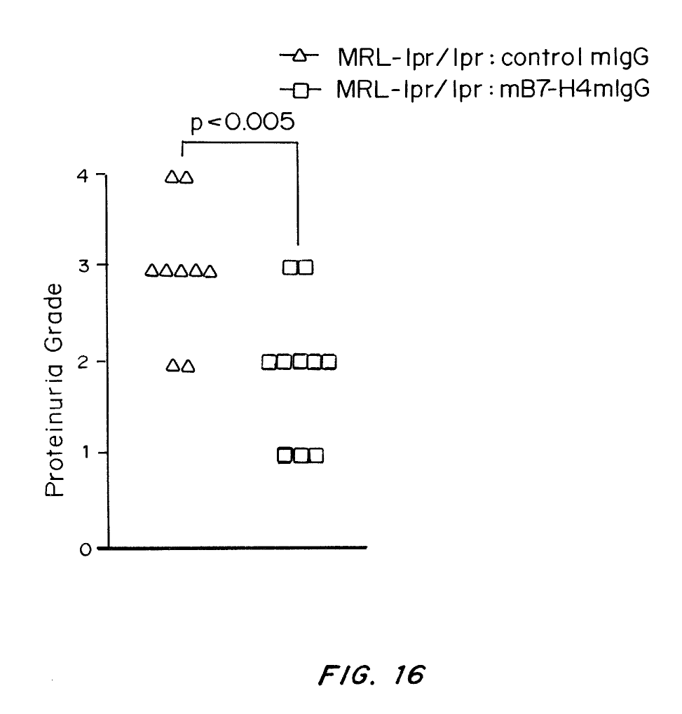

FIG. 15 is a line graph of IgG autoantibody titer (A₄₅₀ₙₘ) versus age (weeks) in MRL-lpr/lpr mice injected with control mIgG plasmid (□) or B7-H4Ig plasmid (▪). FIG. 15 shows that treatment by B7-H4Ig (murine) vector inhibits autoantibodies (anti-DNA) in MRL-lpr/lpr mice. FIG. 16 is a graph of proteinuria grade in MRL-lpr/lpr mice injected with control mIgG plasmid (□) or B7-H4Ig plasmid (□). FIG. 16 shows that treatment by B7-H4Ig (murine) vector inhibits kidney damage in MRL-lpr/lpr mice(1).

Statistical analysis. Statistical analysis was performed with the Mann-Whitney U test for single comparison and ANOVA followed by the Scheffe test for multiple comparisons. In all statistical analyses, significance was accepted at P<0.05.

Those skilled in the art will recognize, or be able to ascertain using no more than routine experimentation, many equivalents to the specific embodiments described herein. Such equivalents are intended to be encompassed by the following claims.

We claim:

1. A pharmaceutical composition comprising a B7-H4 receptor agonist in an amount effective to inhibit or reduce one or more symptoms of an inflammatory response or autoimmune disease or disorder.

2. The pharmaceutical composition of claim 1 wherein the B7-H4 receptor agonist is selected from the group consisting of a polypeptide, small molecule, antibody and an antigen binding fragment thereof.

3. The pharmaceutical composition of claim 2 wherein the polypeptide comprises a fusion protein.

4. The pharmaceutical composition of claim 3 wherein the fusion protein comprises a first fusion partner including all or a part of a B7-H4 extracellular domain fused (i) directly to a second polypeptide or, (ii) optionally, fused to a linker peptide sequence that is fused to the second polypeptide.

5. The pharmaceutical composition of claim 4 wherein the first fusion partner comprises the membrane distal IgV domain and the membrane proximal IgC domain of B7-H4.

6. The pharmaceutical composition of claim 1 in a kit comprising the B7-H4 receptor agonist in a first unit and the pharmaceutically acceptable carrier in a second unit, wherein the units are combined for administration.

7. The pharmaceutical composition of claim 1 wherein the inflammatory response is neutrophil-mediated.

8. The pharmaceutical composition of claim 1 wherein the autoimmune disease or disorder is selected from the group consisting of rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

9. A method for treating or inhibiting one or more symptoms of an inflammatory response in an individual in need thereof comprising administering to the individual a B7-H4 receptor agonist in an amount effective to reduce or inhibit the one or more symptoms of the inflammatory response in the individual.

10. The method of claim 9 wherein the inflammatory response is associated with an autoimmune disease or disorder.

11. The method of claim 10 wherein the individual has an autoimmune disease selected from the group consisting of rheumatoid arthritis, systemic lupus erythematosus, alopecia areata, anklosing spondylitis, antiphospholipid syndrome, autoimmune addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis juvenile, discoid lupus, essential mixed cryoglobulinemia, fibromyalgia fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

12. The method of claim 9 wherein the B7-H4 receptor agonist comprises a B7-H4 polypeptide comprising at least 80% sequence identity to B7-H4 extracellular domain and is capable of suppressing or inhibiting humoral immunity, cellular immunity, or both.

13. The method of claim 12 wherein the B7-H4 receptor agonist comprises an immunoglobin or fragment thereof.

14. The method of claim 13 wherein the immunoglobin or fragment thereof further comprises an immunoglobin Fc region.

15. The method of claim 9 comprising expressing in the individual a nucleic acid encoding a B7-H4 polypeptide comprising at least 80% sequence identity to B7-H4 extracellular domain.

16. The method of claim 15 wherein the B7-H4 polypeptide further comprises an immunoglobin Fc region.
